(19) United States
(12) Patent Application Publication
(10) Pub. No.: US 2009/0186340 A1
(43) Pub. Date: Jul. 23, 2009

(54) Gene Analysis

(75) Inventors: Martin Lennarth Olsson, Lund (SE); Jill Rosalind Storry, Lund (SE); Neil David Avent, Bristol (GB); Tracey Elizabeth Madgett, Bristol (GB)

Correspondence Address: BANNER & WITCOFF, LTD., 1100 13th STREET, N.W., SUITE 1200, WASHINGTON, DC, 20005-4051, US

(73) Assignees: UNIVERSITY OF THE WEST OF ENGLAND, BRISTOL, Bristol (GB); UNIVERSITETSS 1 LUND, BLODCENTRALEN SKANE, Lund (SE)

(21) Appl. No.: 11/663,624

(22) PCT Filed: Sep. 22, 2005
(86) PCT No.: PCT/GB05/03659

§ 371 Date: Feb. 2, 2009

(30) Foreign Application Priority Data
Sep. 22, 2004 (GB) 0421136.3
Mar. 23, 2005 (GB) 0505983.7

(51) Int. Cl. C12Q 1/68; C07H 21/04; C40B 40/08

(52) U.S. Cl. 435/6; 536/24.33; 506/17

(57) ABSTRACT

This invention relates to a series of PCR primers that will allow the simultaneous amplification of regions of the clinically significant ABO and RHD genes.

This invention relates to the field of gene analysis. More particularly, the invention relates to the study of the genotype of a subject in order to perform blood group analysis.

### BACKGROUND

Blood group definition is currently performed using serological techniques for a relatively limited number of clinically significant blood groups. Recent advances have included the determination of blood groups using molecular genetic techniques, but these have only been used in circumscribed situations, for example: the prenatal determination where the isolation of foetal blood for serological investigation would be dangerous or the determination of blood type in multiply transfused patients where serology is difficult because of the admix of patient/donor blood.

Currently large-scale blood group genotyping is not performed due to limitations of molecular-genetic based technologies and the relatively low cost of the current serological testing methodology. However, blood group serology has significant drawbacks. For example, the number of reagents available for testing some blood group antigen specificities is limited or such reagents may not exist. As a consequence, not all blood group antigens are tested for routinely. This can lead to primary alloimmunisation events where the recipients of blood become immunised to the antigens carried on the donated red blood cells. Blood group genotyping of all blood donors would result in more comprehensive blood testing and may result in a reduction in the incidence of alloimmunisations and subsequent transfusion reactions.

The ABO blood group is the most significant of all human blood groups and can cause immediate transfusion reactions, possibly leading to death, when ABO-incompatible blood is transfused. This is because blood group A, B and O individuals have preformed anti-A and/or anti-B in their serum (made to bacterial carbohydrate antigens) that will cross react with red cell A and/or B antigens not found on their own red cells. ABO compatibility is a major cause of transfusion associated morbidity and mortality and every blood donor and patient receiving blood, blood products or solid organ transplants must have their ABO status defined.

Red cell serology is used routinely for defining the ABO status of human red cells utilized in transfusion therapy. Despite this widespread and cheap application of serological techniques, ABO genotyping has some applications in routine Transfusion Medicine. Rare A and B alleles have depressed expression of both sets of antigens (e.g. A3, B3, Aₑₗ, Bₑₗ, A_{X} and B_{X}). These rare variants can be missed by routine automated ABO typing, with some of these potentially being typed as blood group O. Many of these alleles are caused by hybrid ABO genes and can only be classified using molecular genetic techniques. If blood grouping by molecular genetic techniques becomes a frontline replacement to red cell serology, then robust tests for ABO genotype will need to be developed and utilized (Olsson (2001) Blood 98 1584-1593).

The A and B antigens of the ABO histo-blood group system are synthesized by glycosyltransferases encoded by the ABO locus on chromosome 9. The gene encoding the A glycosyltransferase was the first to be isolated, cloned and sequenced (Clausen et al (1990) J. Biol. Chem. 265 1139-1145; Yamamoto et al (1990) Nature 345 229-233). Sequence analysis revealed a coding region of 1062 bp that corresponds to a 41 kDa protein. This coding region was shown subsequently to be distributed over 7 exons (Yamamoto et al (1995) Glycobiology 5 51-58; Bennett et al (1995) Biochem. Biophys. Res. Commun. 211 347) and the gene spans a region of ˜20 kb on 9q34. The consensus coding sequence is the A101 allele and all polymorphisms that affect the specificity and efficacy of the glycosyltransferase are considered mutations of this allele.

Most of the mutations that affect the specificity and/or efficacy of the encoded glycosyltransferase occur in exons 6 and 7. However there are a few important mutations in the earlier exons (Chester & Olsson (2001) Trans. Med. Rev. 11 295-313). Mutations that encode the major alleles are shown in Table 1.

TABLE 1
Selected nucleotide polymorphisms between the major
alleles of the ABO gene located in exons 6 and 7.

| Nucleotide | 261 | 297 | 467 | 526 | 703 | 796 | 802 | 803 | 1060 |
|---|---|---|---|---|---|---|---|---|---|
| A1 (A101) | G | A | C | C | G | C | G | G | C |
| A2 (A201) | — | — | T | — | — | — | — | — | Deletion |
| B (B101) | — | G | — | G | A | A | — | C | — |
| O1 (O01) | Deletion | — | — | — | — | — | — | — | — |
| O1^{ν} (O02) | Deletion | G | — | — | — | — | — | — | — |
| O2 (O03) | — | G | — | G | — | — | A | — | — |

No change is indicated by “—”. Nucleotides that generate a change in the ammo acid coded are shown in bold font. Alternative allele names are shown in parentheses (http://www.bioc.aecom.yu.edu/bgmut.index.htm).

The Rh system is the most polymorphic blood group system and is of significant importance in transfusion medicine. The Rh system is involved in haemolytic transfusion reactions, neonatal haemolytic disease and autoimmune haemolytic anaemia. There are two different, but highly homologous, genes in the Rh system. One gene (RHD) encodes the D polypeptide and the other (RHCE) the CcEe polypeptide. RHD carries the D antigen as the most potent blood group immunogen. This antigen is absent from a relatively large segment (15-17%) of the population (i.e. the Rh-negative phenotype), as a result of RHD gene deletion or other gene alterations. RHCE exists in four allelic forms and each allele determines the expression of two antigens in Ce, ce, cE or CE combination (RHCE is the collective name of the four alleles).

Multiplex (MPX) Polymerase Chain Reaction (PCR) is a variation on the well-known PCR technique, and employs different primer pairs in the same amplification reaction. It has been used in the analysis of blood groups. MPX PCR primers for amplification of Rh D sequences have been previously produced. Avent N D et al. Blood, 1997, 89 2568-77 discloses a multiplex RHD genotyping assay based on amplification of RHD intron 4 and the 3′ non-coding region. Subsequently, six further RHD gene primer sequences have been produced for use in MPX PCR (Maaskant-van Wijk P A et al Transfusion 38, November/December 1998, 1015-1021). In this disclosure, primers were designed to amplify various exons of the RHD gene. It was also indicated that RHD assays should not be dependent on non coding regions of the RHD gene (i.e. introns) and that the technique might be of great value in prenatal RH genotyping. Wagner et al., 1999, Blood, 93, 385-393 disclosed a normal PCR based method involving primers to amplify relatively large PCR products. Due to the size of the products amplified, the PCR primers could not be used in a multiplex PCR method.

The inventors have prepared primers that can be used in multiplex PCR for use in blood group genotyping analysis, in particular, RHD and ABO genotyping analysis. The primers have been identified and selected to amplify fragments of an appropriate size for MPX PCR (in this case they are smaller than 1315 bp) and have also been selected for functionality, that is to say, the selected primers provide good amplification of the desired fragments and are specific to the desired fragments.

### SUMMARY OF THE INVENTION

According to a first aspect of the present invention there is provided a method of RHD genotyping analysis, by multiplex PCR, the method comprising contacting RHD gene nucleic acids from a subject with one or more of the following primer pairs 1,2;3,4 or 4A;5,6;7,8 or 8A;9 or 9A or 10 or 10A or 10B,11 or 11A;12,13;14 or 14A,15 or 15A;16,17;18,19; and 30,31 from the following table (table 2), wherein the primer pairs may comprise the entire sequence shown in the table or the sequence shown in uppercase:

TABLE 2

| Primer | Primer | |
|---|---|---|
| no. | name | Sequence (5′-3′) |
| 1 | 101F | gccgcgaattcactagtgCCATAGAGAGGCCAGCACAA |
| 2 | 198R | ggccgcgggaattcgattTGCCCCTGGAGAACCAC |
| 3 | intlF | gccgcgaattcactagtgTGACGAGTGAAACTCTATCTCGAT |
| 4 | 297R | ggccgcgggaattcgattCCACCATCCCAATACCTGAAC |
| 4A | 296R | ggccgcgggaattcgattAGAAGTGATCCAGCCACCAT |
| 5 | 303F | gccgcgaattcactagtgTCCTGGCTCTCCCTCTCT |
| 6 | 397R | ggccgcgggaattcgattGTTGTCTTTATTTTTCAAAACCCT |
| 7 | 403F | gccgcgaattcactagtgGCTCTGAACTTTCTCCAAGGACT |
| 8 | 499R | ggccgcgggaattcgattCAAACTGGGTATCGTTGCTG |
| 8A | 498R | ggccgcgggaattcgattATTCTGCTCAGCCCAAGTAG |
| 9 | 502F | gccgcgaattcactagtgCTTTGAATTAAGCACTTCACAGA |
| 9A | 503F | gccgcgaattcactagtgTTGAATTAAGCACTTCACAGAGCA |
| 10 | 5Aluint4F | gccgcgaattcactagtgAAGGACTATCAGGCCACG |
| | (RoHar) | |
| 10A | RoHar4 | gccgcgaattcactagtgCTGAAAGGAGGGAAACGGAC |
| 10B | RoHar8 | gccgcgaattcactagtgGGGCAGTGAGCTTGATAGTAGG |
| 11 | 599R | ggccgcgggaattcgattCACCTTGCTGATCTTCCC |
| 11A | 598R | ggccgcgggaattcgattTGTGACCACCCAGCATTCTA |
| 12 | 601F | gccgcgaattcactagtgAGTAGTGAGCTGGCCCATCA |
| 13 | 697R | ggccgcgggaattcgattCTTCAGCCAAAGCAGAGGAG |
| 14 | 702F | gccgcgaattcactagtgCTGGGACCTTGTTAGAAATGCTG |
| 14A | 701F | gccgcgaattcactagtgACAAACTCCCCGATGATGTGAGTG |
| 15 | 799R | ggccgcgggaattcgattCAAGGTAGGGGCTGGACAG |
| 15A | 798R | ggccgcgggaattcgattGAGGCTGAGAAAGGTTAAGCCA |
| 16 | 801F | gccgcgaattcactagtgCTGGAGGCTCTGAGAGGTTGAG |
| 17 | 899R | ggccgcgggaattcgattGGCAATGGTGGAAGAAAGG |
| 18 | 901F | gccgcgaattcactagtgACTGTCGTTTTGACACACAAT |
| 19 | 998R | ggccgcgggaattcgattTGTCACCCGCATGTCAG |
| 30 | 1001F | gccgcgaattcactagtgCAAGAGATCAAGCCAAAATCAGT |
| 31 | 1097R | ggccgcgggaattcgattGTGGTACATGGCTGTATTTTATTG |
| 32 | MAPH-rev | gccgcgaattcactagtg |
| 33 | MAPH-forw | ggccgcgggaattcgatt |

and amplifying the RHD gene nucleic acids. Each of the primers indicated in the Table comprises a 5′ MAPH tag (the first 18 nucleotides of the primer sequences shown in lower case) and a gene-specific sequence (shown in upper case). The MAPH tag is used to assist in the amplification of the nucleic acids. Specifically, once the RHD gene nucleic acids have been PCR amplified using the primers, primers to the MAPH tags (32 and 33) are used to further amplify the sequences. Preferably, both amplification steps are performed simultaneously. As will be appreciated by those skilled in the art, primers without the 5′ MAPH tag (primer sequences represented by the sequence in uppercase only) can be used in the method of the invention in order to amplify the RHD gene nucleic acids. Alternatively, the primer sequences can comprise different tag sequences to the MAPH tags indicated in the table.

The method of the invention is advantageous because it allows the simultaneous amplification of ten regions, exons 1 to 10 of the highly clinically significant RHD gene. This includes most known RID alleles, including the clinically significant partial and weak D variants. In particular, it includes exon 10, in which there is a mutation that results in the Del phenotype recently described in Gassner C, Doescher A, Drnovsek T D, Rozman P, Eicher N I, Legler T J, Lukin S, Garritsen H, Kleinrath T, Egger B, Ehling R, Kormoczi G F, Kilga-Nogler S, Schoenitzer D, Petershofen E K. (2005) Transfusion 45(4) 527-538 Presence of RHD in serologically D−, C/E+ individuals: a European multicenter study. The method permits even more comprehensive blood testing and should result in a reduction in the incidence of alloimmunisations and subsequent transfusion reactions.

The method is also advantageous in that it can distinguish some common partial D phenotypes that are caused by hybrid RHD-RHCE genes including the DV and DVI phenotypes. These phenotypes will lack predicted fragments following amplification. DVI phenotypes are relatively common, occurring once in every 4000 individuals of Western European descent There are at least eight different genetic bases associated with the DV phenotype and at least four different genetic bases associated with the DVI phenotype. All known DV phenotypes can be differentiated following subsequent further analysis of the MPX products. DVI phenotype individuals lack a large number of D epitopes and can become alloimmunised to the RHD antigen by transfusion or pregnancy. In the UK DVI mothers are deliberately typed as D-negative, so they receive anti-D to avoid alloimmunisation. However, if blood donors of DVI phenotype are typed as D-negative, this blood may be transfused to “true” D-negative individuals and alloimmunisation may result. Genotyping using the assay of the invention would identify DVI persons and they can be excluded from the donor population for transfusion to D-negative individuals.

The method is further advantageous in that it can be used for analysis of adult donor subjects. This is important in connection with subjects who receive frequent transfusions, for example, those with sickle cell anaemia.

The DHAR phenotype is associated with a hybrid RHCE-RHD gene where exon 5 of RHCE is replaced by RHD (Beckers E A et al., Br J Haematol. 1996 March; 92(3):751-7.). DHAR red cells express a small but significant number of D epitopes. Using conventional serological techniques these individuals may type as Rh D-negative and their blood could potentially be transfused into D-negative individuals. These individuals may become immunised. DHAR is a very rare blood group. The assay of the invention permits the detection of the DHAR phenotype.

At least one of the primers used in the method is preferably labelled to allow detection of the amplified product. Suitable labels are well known to those skilled in the art. For example, it may be desirable to label one of the primers with 6-FAM.

The nucleic acids used in this and subsequent aspects of the invention may be derived from any appropriate source, such as, but not limited to blood, a buccal smear, urine, amniotic fluid. The nucleic acids are preferably derived from blood.

The blood may be utilized in any known manner, for example, ex vivo. In particular, the method of the invention may be performed on blood directly removed from an individual, for example, a patient requiring a blood transfusion or may be performed on a sample of blood to be delivered to an individual, for example, blood from a blood donation.

The nucleic acid is preferably DNA, most preferably genomic DNA.

The annealing temperature may be from 54-63° C. Preferably the annealing temperature is about 60° C. Most preferably the annealing temperature is 60° C.

The method of the invention may be combined with other MPX PCR methods to genotype other blood group genes. For example the method of the invention may be combined with MPX PCRs for the ABO/MNS/P1/RHCE/LU (Lutheran)/KE(Kell)/LE(Lewis)/FY(Duffy)/JK(Kidd)/DI(Diego)/YT(Cartwright)/XG/SC(Scianna)/DO(Dombrock)/CO(Colton)/LW/CH/RG(Chido/Rodgers)/Hh/XK/GE(Gerbich)/CROM(Cromer)/KN(Knops)/IN(Indian)/OK/RAPH/JMH(JohnMiltonHagen)/IGNT/P and/or GIL systems and/or any other blood group system that is known or becomes known.

Nucleic acids amplified by the method of the invention may be detected using any suitable method. For example, the amplified nucleic acid may be hybridised with a suitable nucleic acid probe specific for the sequence to be detected. Suitable nucleic acid probes can be provided in a format such as a gene chip. Preferably, the gene chip includes nucleic acid probes which hybridise to nucleic acids specific for other blood group genotypes.

In a preferred method of the invention the RHD gene nucleic acids are contacted with one or more of the following primer pairs: 1,2;3,4;5,6;7,8;9 or 10,11;12,13;14,15; and 18,19, preferably all of those primer pairs.

In an alternative preferred embodiment, the RHD gene nucleic acids are contacted with one or more of the following primer pairs 1,2;3,4A;5,6;7,8A;9A or 10A or 10B,11A;12,13;14A,15A; 16,17; 18,19; and 30,31, preferably all of those primer pairs.

Most preferably the RHD gene nucleic acids are contacted with all the following primer pairs 1,2;3,4 or 4A;5,6;7,8 or 8A;9 or 9A or 10 or 10A or 10B,11 or 11A;12,13;14 or 14A,15 or 15A;16,17;18,19; and 30,31.

According to a second aspect of the invention there is provided a method of RHD genotyping analysis, by multiplex PCR, the method comprising contacting RHD gene nucleic acids derived from blood from a subject with at least one primer selected from the following table (table 2A) wherein the primer may comprise the entire sequence shown in the table or the sequence shown in uppercase:

TABLE 2A

| Primer | Primer | |
|---|---|---|
| no. | name | Sequence (5′-3′) |
| 1 | 101F | gccgcgaattcactagtgCCATAGAGAGGCCAGCACAA |
| 4 | 297R | ggccgcgggaattcgattCCACCATCCCAATACCTGAAC |
| 4A | 296R | ggccgcgggaattcgattAGAAGTGATCCAGCCACCAT |
| 5 | 303F | gccgcgaattcactagtgTCCTGGCTCTCCCTCTCT |
| 6 | 397R | ggccgcgggaattcgattGTTGTCTTTATTTTTCAAAACCCT |
| 7 | 403F | gccgcgaattcactagtgGCTCTGAACTTTCTCCAAGGACT |
| 8A | 498R | ggccgcgggaattcgattATTCTGCTCAGCCCAAGTAG |
| 9 | 502F | gccgcgaattcactagtgCTTTGAATTAAGCACTTCACAGA |
| 9A | 503F | gccgcgaattcactagtgTTGAATTAAGCACTTCACAGAGCA |
| 10 | 5Aluint4F | gccgcgaattcactagtgAAGGACTATCAGGCCACG |
| | (RoHar) | |
| 10A | RoHar4 | gccgcgaattcactagtgCTGAAAGGAGGGAAACGGAC |
| 10B | RoHar8 | gccgcgaattcactagtgGGGCAGTGAGCTTGATAGTAGG |
| 11 | 599R | ggccgcgggaattcgattCACCTTGCTGATCTTCCC |
| 11A | 598R | ggccgcgggaattcgattTGTGACCACCCAGCATTCTA |
| 12 | 601F | gccgcgaattcactagtgAGTAGTGAGCTGGCCCATCA |
| 13 | 697R | ggccgcgggaattcgattCTTCAGCCAAAGCAGAGGAG |
| 14 | 702F | gccgcgaattcactagtgCTGGGACCTTGTTAGAAATGCTG |
| 14A | 701F | gccgcgaattcactagtgACAAACTCCCCGATGATGTGAGTG |
| 15 | 799R | ggccgcgggaattcgattCAAGGTAGGGGCTGGACAG |
| 15A | 798R | ggccgcgggaattcgattGAGGCTGAGAAAGGTTAAGCCA |
| 17 | 899R | ggccgcgggaattcgattGGCAATGGTGGAAGAAAGG |
| 18 | 901F | gccgcgaattcactagtgACTGTCGTTTTGACACACAAT |
| 19 | 998R | ggccgcgggaattcgattTGTCACCCGCATGTCAG |
| 31 | 1097R | ggccgcgggaattcgattGTGGTACATGGCTGTATTTTATTG |

and amplifying the RHD gene nucleic acids. As will be appreciated by those skilled in the art, a pair of primers needs to be used to obtain amplification. Both primers may be selected from table 2A or one of the primers can be selected from table 2A and used with any suitable second primer, for example, a primer from table 2 or any other suitable primer. The pair of primers may be used alone or with any other primers. Preferably, the method comprises contacting the RHD gene nucleic acids with one or more of the following primer pairs: 1,2;3,4 or 4A;5,6;7,8 or 8A;9 or 9A or 10 or 10A or 10B, 11 or 11A;12,13;14 or 14A,15 or 15A;16,17;18,19; and 30,31.

In an alternative preferred embodiment, the method comprises contacting the RHD gene nucleic acids with one or more of the following primer pairs: 1 and 2; 5 and 6; 10 and 11; 12 and 13; 14 and 15 and 18 and 19.

In a further alternative preferred embodiment, the method comprises contacting the RHD gene nucleic acids with one or more of the following primer pairs: 1,2;3, 4A;5,6;7,8A; 9A or 10A or 10B,11A;12,13;14A, 15A;16,17;18,19; and 30,31.

In this and subsequent methods of the invention, primer pairs may be used individually or in combination to amplify, for example, one, several or all exons of interest.

As indicated above for the first aspect of the present invention, each of the primers indicated in table 2 comprises a 5′ MAPH tag (the first 18 nucleotides of the primer sequences shown in lower case) and a gene-specific sequence. As will be appreciated by those skilled in the art, primers without the 5′ MAPH tag (primer sequences represented by the sequence in uppercase only) can be used in the method of the invention in order to amplify the RHD gene nucleic acids. Alternatively, the primer sequences can comprise different tag sequences to the MAPH tags indicated in the table.

According to a third aspect of the invention there is provided a method of ABO genotyping analysis, by multiplex PCR, the method comprising contacting ABO gene nucleic acids from a subject with one or more of the following primer pairs 20,21; 22,23; 24 or 24A,25; 26,27 and 28,29 from the following table (table 3), wherein the primer pairs may comprise the entire sequence shown in the table or the sequence shown in uppercase:

TABLE 3

| Primer | | |
|---|---|---|
| no. | Primer name | Sequence (5′-3′) |
| 20 | int1 − 49f | gccgcgaattcactagtgGTGAGAGAAGGAGG |
| | | GTGAG |
| 21 | int2 + 62r | ggccgcgggaattcgattATTGGCTGCTGTGG |
| | | TCA |
| 22 | int3 − 33f | gccgcgaettcactagtgcCTGCTCCTAGACT |
| | | AAACTTC |
| 23 | int4 + 52r | ggccgcgggaattcgattAAGGGAGGCACTGA |
| | | CATTA |
| 24 | int5 + 44f | gccgcgaattcactagtgCTGCCAGCTCCATG |
| | | TGAC |
| 24A | int5 + 367f | gccgcgaattcactagtgGATTTGCCCGGTTG |
| | | GAGTC |
| 25 | int6 + 31r | ggccgcgggaattcgattAGTCACTCGCCACT |
| | | GCC |
| 26 | ABO432f | gccgcgaattcactagtgcCACCGTGTCCACT |
| | | ACTATG |
| 27 | ABO766r | ggccgcgggaattcgattTGTAGGCCTGGGAC |
| | | TGG |
| 28 | ABO723f | gccgcgaattcactagtgGGAGGCCTTCACCT |
| | | ACG |
| 29 | ABO1147r | ggccgcgggaattcgattCAGAGTTTACCCGT |
| | | TCTGC |

and amplifying the ABO gene nucleic acids. Preferably the ABO gene nucleic acids are contacted with all of the primer pairs mentioned.

Each of the primers indicated in table 3 comprises a 5′ MAPH tag (the first 18 nucleotides of the primer sequences shown in lower case) and a gene-specific sequence (shown in upper case). The MAPH tag is used to assist in the amplification of the nucleic acids. Specifically, once the ABO gene nucleic acids have been PCR amplified using the primers, primers to the MAPH tags are used to further amplify the sequences. Preferably, both amplification steps are performed simultaneously. As will be appreciated by those skilled in the art, primers without the 5′ MAPH tag (primer sequences represented by the sequence in uppercase only) can be used in the method of the invention in order to amplify the ABO gene nucleic acids. Alternatively, the primer sequences can comprise different tag sequences to the MAPH tags indicated in the table.

These primers amplify ABO exons 2, 4, 6, and 7 in a gene-specific manner such that allele specificity is determined by the use of oligonucleotide probes specific for a given allele. The primer sequences have been selected to deliberately exclude any known ABO nucleotide polymorphism, so as to be gene but not allele specific. Amplification of the ABO gene by this primer set permits the identification by sequence-specific oligonucleotide probes of all known ABO variants.

The blood may be utilized in any known manner, for example, ex vivo. In particular, the method of the invention may be performed on blood directly removed from an individual, for example, a patient requiring a blood transfusion or may be performed on a sample of blood to be delivered to an individual, for example, blood from a blood donation.

The nucleic acid is preferably DNA, more preferably genomic DNA.

The annealing temperature may be from 54-63° C. Preferably the annealing temperature is about 57° C. Most preferably the annealing temperature is 57° C.

The method of the third aspect of the invention may be combined with other MPX PCR methods to genotype other blood group genes. For example the method of the invention may be combined with MPX PCRs for the RHD//MNS/P1/RHCE/LU (Lutheran)/KE(Kell)/LE(Lewis)/FY(Duffy)/JK(Kidd)/DI(Diego)/YT(Cartwright)/XG/SC(Scianna)/DO(Dombrock)/CO(Colton)/LW/CH/RG(Chido/Rodgers)/Hh/XK/GE(Gerbich)/CROM(Cromer)/KN(Knops)/IN(Indian)/OK/RAPH/JMH(JohnMiltonHagen)/IGNT/P and/or GIL systems and/or any other blood group system that is known or becomes known.

Nucleic acids amplified by the method of the third aspect of the invention may be detected as indicated above.

Preferably, the method comprises contacting ABO gene nucleic acids derived from blood from a subject with one or more, preferably all, of the following primer pairs 20,21; 22,23; 24,25; 26,27 and 28,29.

Alternatively, the method comprises contacting ABO gene nucleic acids derived from blood from a subject with one or more, preferably all, of the following primer pairs 20,21; 22,23; 24A,25; 26,27 and 28,29.

According to a fourth aspect of the present invention, there is provided a method of ABO genotyping analysis, by multiplex PCR, the method comprising contacting ABO gene nucleic acids derived from blood from a subject with at least one primer selected from the following table (table 3), wherein the primer may comprise the entire sequence shown in the table or the sequence shown in uppercase:

TABLE 3

| Primer | | |
|---|---|---|
| no. | Primer name | Sequence (5′-3′) |
| 20 | int1 − 49f | gccgcgaattcactagtgGTGAGAGAAGGAGG |
| | | GTGAG |
| 21 | int2 + 62r | ggccgcgggaattcgattATTGGCTGCTGTGG |
| | | TCA |
| 22 | int3 − 33f | gccgcgaattcactagtgcCTGCTCCTAGACT |
| | | AAACTTC |
| 23 | int4 + 52r | ggccgcgggaattcgattAAGGGAGGCACTGA |
| | | CATTA |
| 24 | int5 − 44f | gccgcgaattcactagtgCTGCCAGCTCCATG |
| | | TGAC |
| 24A | int5 − 367f | gccgcgaattcactagtgGATTTGCCCGGTTC |
| | | GGAGTC |
| 25 | int6 + 31r | ggccgcgggaattcgattAGTCACTCGCCACT |
| | | GCC |
| 26 | ABO432f | gccgcgaattcactagtgcCACCGTGTCCACT |
| | | ACTATG |
| 27 | ABO766r | ggccgcgggaattcgattTGTAGGCCTGGGAC |
| | | TGG |
| 28 | ABO723f | gccgcgaattcactagtgGGAGGCCTTCACCT |
| | | ACG |
| 29 | ABO1147r | ggccgcgggaattcgattCAGAGTTTACCCGT |
| | | TCTGC |

and amplifying the ABO gene nucleic acids. As will be appreciated by those skilled in the art, a pair of primers needs to be used to obtain amplification. Both primers may be selected from table 3 or one of the primers can be selected from table 3 and used with any suitable second primer. The pair of primers may be used alone or with any other primers. Preferably, the method comprises contacting the ABO gene nucleic acids with one or more of the following primer pairs: 20 and 21; 22 and 23; 24 or 24A and 25; 26 and 27; 28 and 29.

In a preferred embodiment, the method comprises contacting the ABO gene nucleic acids with one or more of the following primer pairs: 20 and 21; 22 and 23; 24 and 25; 26 and 27; 28 and 29.

In an alternative embodiment, the method comprises contacting the ABO gene nucleic acids with one or more of the following primer pairs: 20 and 21; 22 and 23; 24A and 25; 26 and 27; 28 and 29.

According to a fifth aspect of the invention, there is provided a method of ABO and RHD genotyping analysis, by multiplex PCR, the method comprising contacting ABO gene and RHD gene nucleic acids derived from blood from a subject with one or more of the following primer pairs 1,2; 3,4 or 4A; 5,6; 7,8 or 8A; 9 or 9A or 10 or 10A or 10B,11 or 11A; 12,13; 14 or 14A,15 15A; 16,17; 18,19; 20,21; 22,23; 24 or 24A,25; 26,27; 28,29; and 30,31 from the following table (table 4), wherein the primer pairs may comprise the entire sequence shown in the table or the sequence shown in uppercase:

TABLE 4

| Primer | | |
|---|---|---|
| no. | Primer name | Sequence (5′-3′) |
| 1 | 101F | gccgcgaattcactagtgCCATAGAGAGGCCA |
| | | GCACAA |
| 2 | 198R | ggccgcgggaattcgattTGCCCCTGGAGAAC |
| | | CAC |
| 3 | int1F | gccgcgaattcactagtgTGACGAGTGAAACT |
| | | CTATCTCGAT |
| 4 | 297R | ggccgcgggaattcgattCCACCATCCCAATA |
| | | CCTGAAC |
| 4A | 296R | ggccgcgggaattcgattAGAAGTGATCCAGC |
| | | CACCAT |
| 5 | 303F | gccgcgaattcactagtgTCCTGGCTCTCCCT |
| | | CTCT |
| 6 | 397R | ggccgcgggaattcgattGTTGTCTTTATTTT |
| | | TCAAAACCCT |
| 7 | 403F | gccgcgaattcactagtgGCTCTGAACTTTCT |
| | | CCAAGGACT |
| 8 | 499R | ggccgcgggaattcgattCAAACTGGGTATCG |
| | | TTGCTG |
| 8A | 498R | ggccgcgggaattcgattATTCTGCTCAGCCC |
| | | AAGTAG |
| 9 | 502F | gccgcgaattcactagtgCTTTGAATTAAGCA |
| | | CTTCACAGA |
| 9A | 503F | gccgcgaattcactagtgTTGAATTAAGCACT |
| | | TCACAGAGCA |
| 10 | 5Aluint4F | gccgcgaattcactagtgAAGGACTATCAGGC |
| | (RoHar) | CACG |
| 10A | RoHar4 | gccgcgaattcactagtgCTGAAAGGAGGGAA |
| | | ACGGAC |
| 10B | RoHar8 | gccgcgaattcactagtgGGGCAGTGAGCTTG |
| | | ATAGTAGG |
| 11 | 599R | ggccgcgggaattcgattCACCTTGCTGATCT |
| | | TCCC |
| 11A | 598R | ggccgcgggaattcgattTGTGACCACCCAGC |
| | | ATTCTA |
| 12 | 601F | gccgcgaattcactagtgAGTAGTGAGCTGGC |
| | | CCATCA |
| 13 | 697R | ggccgcgggaattcgattCTTCAGCCAAAGCA |
| | | GAGGAG |
| 14 | 702F | gccgcgaattcactagtgCTGGGACCTTGTTA |
| | | GAAATGCTG |
| 14A | 701F | gccgcgaattcactagtgACAAACTCCCCGAT |
| | | GATGTGAGTG |
| 15 | 799R | ggccgcgggaattcgattCAAGGTAGGGGCTG |
| | | GACAG |
| 15A | 798R | ggccgcgggaattcgattGAGGCTGAGAAAGG |
| | | TTAAGCCA |
| 16 | 801F | gccgcgaattcactagtgCTGGAGGCTCTGAG |
| | | AGGTTGAG |
| 17 | 899R | ggccgcgggaattcgattGGCAATGGTGGAAG |
| | | AAAGG |
| 18 | 901F | gccgcgaattcactagtgACTGTCGTTTTGAC |
| | | ACACAAT |
| 19 | 998R | ggccgcgggaattcgattTGTCACCCGCATGT |
| | | CAG |
| 30 | 1001F | gccgcaattcactagtgCAAGAGATCAAGCCA |
| | | AAATCAGT |
| 31 | 1097R | ggccgcgggaattcgattGTGGTACATGGCTG |
| | | TATTTTATTG |
| 20 | int1 − 49f | gccgcgaattcactagtgGTGAGAGAAGGAGG |
| | | GTGAG |
| 21 | int2 + 62r | ggccgcgggaattcgattATTGGCTGCTGTGG |
| | | TCA |
| 22 | int3 − 33f | gccgcgaattcactagtgcCTGCTCCTAGACT |
| | | AAACTTC |
| 23 | int4 + 52r | ggccgcgggaattcgattAAGGGAGGCACTGA |
| | | CATTA |
| 24 | int5 − 44f | gccgcgaattcactagtgCTGCCAGCTCCATG |
| | | TGAC |
| 24A | int5 − 367f | gccgcgaattcactagtgGATTTGCCCGGTTG |
| | | GAGTC |
| 25 | int6 + 31r | ggccgcgggaattcgattAGTCACrCGCCACT |
| | | GCC |
| 26 | ABO432f | gccgcgaattcactagtgcCACCGTGTCCACT |
| | | ACTATG |
| 27 | ABO766r | ggccgcgggaattcgattTGTAGGCCTGGGAC |
| | | TGG |
| 28 | ABO723f | gccgcgaattcactagtgGGAGGCCTTCACCT |
| | | ACG |
| 29 | ABO1147r | ggccgcgggaattcgattCAGAGTTTACCCGT |
| | | TCTGC |

and amplifying the RHD and ABO gene nucleic acids. Preferably the nucleic acids are contacted with all the pairs mentioned above.

As indicated above for the previous aspects of the present invention, each of the primers indicated in table 4 comprises a 5′ MAPH tag (the first 18 nucleotides of the primer sequences shown in lower case) and a gene-specific sequence (shown in upper case). As will be appreciated by those skilled in the art, primers without the 5′ MAPH tag (primer sequences represented by the sequence in uppercase only) can be used in the method of the invention in order to amplify the RHD and ABO gene nucleic acids. Alternatively, the primer sequences can comprise different tag sequences to the MAPH tags indicated in table 4.

Preferably, the method comprises contacting the ABO gene and RHD gene nucleic acids with one or more, preferably all, of the following primer pairs: 1,2; 3,4; 5,6; 7,8; 9 or 10,11; 12,13; 14,15; 18,19; 20,21; 22,23; 24,25; 26,27 and 28,29.

Alternatively, the method comprises contacting the ABO gene and RHD gene nucleic acids with one or more, preferably all, of the following primer pairs: 1,2; 3,4; 5,6; 7,8A; 9A or 10A or 10B,11A; 12,13; 14A,15A; 16, 17;18,19; 20,21; 22,23; 24A,25; 26,27; 28,29; and 30,31.

The blood may be utilized in any known manner, for example, ex vivo. In particular, the method of the invention may be performed on blood directly removed from an individual, for example, a patient requiring a blood transfusion or may be performed on a sample of blood to be delivered to an individual, for example, blood from a blood donation.

The nucleic acid is preferably DNA, more preferably genomic DNA.

The annealing temperature may be from 54-63° C. Preferably the annealing temperature is about 60° C. or about 57° C. Most preferably the annealing temperature is 60° C.

The method of the fifth aspect of the invention may be combined with other MPX PCR methods to genotype other blood group genes. For example the method of the invention may be combined with MPX PCRs for the MNS/P1/RHCE/LU (Lutheran)/KE(Kell)/LE(Lewis)/FY(Duffy)/JK(Kidd)/DI(Diego)/YT(Cartwright)/XG/SC(Scianna)/DO(Dombrock)/CO(Colton)/LW/CH/RG(Chido/Rodgers)/Hh/XK/GE(Gerbich)/CROM(Cromer)/KN(Knops)/IN(Indian)/OK/RAPH/JMH(JohnMiltonHagen)/IGNT/P and/or GIL systems and/or any other blood group system that is known or becomes known.

Nucleic acids amplified by the method of the fifth aspect of the invention may be detected as indicated above.

According to a sixth aspect of the invention, there is provided a method of ABO and RHD genotyping analysis, by multiplex PCR, the method comprising contacting ABO gene and RHD gene nucleic acids derived from blood from a subject with one or more primer from the following table (table 4A), wherein the primer may comprise the entire sequence shown in the table or the sequence shown in uppercase:

TABLE 4A

| Primer | | |
|---|---|---|
| no. | Primer name | Sequence (5′-3′) |
| 1 | 101F | gccgcgaattcactagtgCCATAGAGAGGCCA |
| | | GCACAA |
| 4 | 297R | ggccgcgggaattcgattCCACCATCCCAATA |
| | | CCTGAAC |
| 4A | 296R | ggccgcgggaattcgattAGAAGTGATCCAGC |
| | | CACCAT |
| 5 | 303F | gccgcgaattcactagtgTCCTGGCTCTCCCT |
| | | CTCT |
| 6 | 397R | ggccgcgggaattcgattGTTGTCTTTATTTT |
| | | TCAAAACCCT |
| 7 | 403F | gccgcgaattcactagtgGCTCTGAACTTTCT |
| | | CCAAGGACT |
| 8A | 498R | ggccgcgggaattcgattATTCTGCTCAGCCC |
| | | AAGTAG |
| 9 | 502F | gccgcgaattcactagtgCTTTGAATTAAGCA |
| | | CTTCACAGA |
| 9A | 503F | gccgcgaattcactagtgTTGAATTAAGCACT |
| | | TCACAGAGCA |
| 10 | 5Aluint4F | gccgcgaattcactagtgAAGGACTATCAGGC |
| | (RoHar) | CACG |
| 10A | RoHar4 | gccgcgaattcactagtgCTGAAAGGAGGGAA |
| | | ACGGAC |
| 10B | RoHar8 | gccgcgaattcactagtgGGGCAGTGAGCTTG |
| | | ATAGTAGG |
| 11 | 599R | ggccgcgggaattcgattCACCTTGCTGATCT |
| | | TCCC |
| 11A | 598R | ggccgcgggaattcgattTGTGACCACCCAGC |
| | | ATTCTA |
| 12 | 601F | gccgcgaattcactagtgAGTAGTGAGCTGGC |
| | | CCATCA |
| 13 | 697R | ggccgcgggaattcgattCTTCAGCCAAAGCA |
| | | GAGGAG |
| 14 | 702F | gccgcgaattcactagtgCTGGGACCTTGTTA |
| | | GAAATGCTG |
| 14A | 701F | gccgcgaattcactagtgACAAACTCCCCGAT |
| | | GATGTGAGTG |
| 15 | 799R | ggccgcgggaattcgattCAAGGTAGGGGCTG |
| | | GACAG |
| 15A | 798R | ggccgcgggaattcgattGAGCTGAGAAAGGT |
| | | TAAGCCA |
| 17 | 899R | ggccgcgggaattcgattGGCAATGGTGGAAG |
| | | AAAGG |
| 18 | 901F | gccgcgaattcactagtgACTGTCGTTTTGAC |
| | | ACACAAT |
| 19 | 998R | ggccgcgggaattcgattTGTCACCCGCATGT |
| | | CAG |
| 31 | 1097R | ggccgcgggaattcgattGTGGTACATGGCTG |
| | | TATTTTATTG |
| 20 | int1 − 49f | gccgcgaattcactagtgGTGAGAGAAGGAGG |
| | | GTGAG |
| 21 | int2 + 62r | ggccgcgggaattcgattATTGGCTGCTGTGG |
| | | TCA |
| 22 | int3 − 33f | gccgcgaattcactagtgcCTGCTCCTAGACT |
| | | AAACTTC |
| 23 | int4 + 52r | ggccgcgggaattcgattAAGGGAGGCACTGA |
| | | CATTA |
| 24 | int5 − 44f | gccgcgaattcactagtgCTGCCAGCTCCATG |
| | | TGAC |
| 24A | int5 − 367f | gccgcgaattcactagtgGATTTGCCCGGTTG |
| | | GAGTC |
| 25 | int6 + 31r | ggccgcgggaattcgattAGTCACTCGCCACT |
| | | GCC |
| 26 | ABO432f | gccgcgaattcactagtgcCACCGTGTCCACT |
| | | ACTATG |
| 27 | ABO766r | ggccgcgggaattcgattTGTAGGCCTGGGAC |
| | | TGG |
| 28 | ABO723f | gccgcgaattcactagtgGGAGGCCTTCACCT |
| | | ACG |
| 29 | ABO1147r | ggccgcgggaattcgattCAGAGTTTACCCGT |
| | | TCTGC |

and amplifying the RHD and ABO gene nucleic acids. As will be appreciated by those skilled in the art, a pair of primers needs to be used to obtain amplification. Both primers may be selected from table 4A or one of the primers can be selected from table 4A and used with any suitable second primer, for example a primer from table 4 or any other suitable primer. The pair of primers may be used alone or with any other primers.

Preferably the method comprises contacting ABO gene and RHD gene nucleic acids with one or more of the following primer pairs: 1,2; 3,4; 5,6; 7,8; 9 or 10,11; 12,13; 14,15; 18,19; 20,21; 22,23; 24,25; 26,27 and 28,29.

Alternatively, the method comprises contacting ABO gene and RHD gene nucleic acids with one or more of the following primer pairs: 1,2; 3,4; 5,6; 7,8A; 9A or 10A or 10B,11A; 12,13; 14A,15A; 18,19; 20,21; 22,23; 24A,25; 26,27; 28,29; and 30,31.

According to a seventh aspect of the invention there are provided one or more of the following PCR primers, wherein the primers may comprise the entire sequence shown in the table or the sequence shown in uppercase:

TABLE 4A

| Primer | | |
|---|---|---|
| no. | Primer name | Sequence (5′-3′) |
| 1 | 101F | gccgcgaattcactagtgCCATAGAGAGGCCA |
| | | GCACAA |
| 4 | 297R | ggccgcgggaattcgattCCACCATCCCAATA |
| | | CCTGAAC |
| 4A | 296R | ggccgcgggaattcgattAGAAGTGATCCAGC |
| | | CACCAT |
| 5 | 303F | gccgcgaattcactagtgTCCTGGCTCTCCCT |
| | | CTCT |
| 6 | 397R | ggccgcgggaattcgattGTTGTCTTTATTTT |
| | | TCAAAACCCT |
| 7 | 403F | gccgcgaattcactagtgGCTCTGAACTTTCT |
| | | CCAAGGACT |
| 8A | 498R | ggccgcgggaattcgattATTCTGCTCAGCCC |
| | | AAGTAG |
| 9 | 502F | gccgcgaattcactagtgCTTTGAATTAAGCA |
| | | CTTCACAGA |
| 9A | 503F | gccgcgaattcactagtgTTGAATTAAGCACT |
| | | TCACAGAGCA |
| 10 | 5Aluint4F | gccgcgaattcactagtgAAGGACTATCAGGC |
| | (RoHar) | CACG |
| 10A | RoHar4 | gccgcgaattcactagtgCTGAAAGGAGGGAA |
| | | ACGGAC |
| 10B | RoHarB | gccgcgaattcactagtgGGGCAGTGAGCTTG |
| | | ATAGTAGG |
| 11 | 599R | ggccgcgggaattcgattCACCTTGCTGATCT |
| | | TCCC |
| 11A | 598R | ggccgcgggaattcgattTGTGACCACCCAGC |
| | | ATTCTA |
| 12 | 601F | gccgcgaattcactagtgAGTAGTGAGCTGGC |
| | | CCATCA |
| 13 | 697R | ggccgcgggaattcgattCTTCAGCCAAAGCA |
| | | GAGGAG |
| 14 | 702F | gccgcgaattcactagtgCTGGGACCTTGTTA |
| | | GAAATGCTG |
| 14A | 701F | gccgcgaattcactagtgACAAACTCCCCGAT |
| | | GATGTGAGTG |
| 15 | 799R | ggccgcgggaattcgattCAAGGTAGGGGCTG |
| | | GACAG |
| 15A | 798R | ggccgcgggaattcgattGAGGCTGAGAAAGG |
| | | TTAAGCCA |
| 17 | 899R | ggccgcgggaattcgattGGCAATGGTGGAAG |
| | | AAAGG |
| 18 | 901F | gccgcgaattcactagtgACTGTCGTTTTGAC |
| | | ACACAAT |
| 19 | 998R | ggccgcgggaattcgattTGTCACCCGCATGT |
| | | CAG |
| 31 | 1097R | ggccgcgggaattcgattGTGGTACATGGCTG |
| | | TATTTTATTG |
| 20 | int1 − 49f | gccgcgaattcactagtgGTGAGAGAAGGAGG |
| | | GTGAG |
| 21 | int2 + 62r | ggccgcgggaattcgattATTGGCTGCTGTGG |
| | | TCA |
| 22 | int3 − 33f | gccgcgaattcactagtgcCTGCTCCTAGACT |
| | | AAACTTC |
| 23 | int4 + 52r | ggccgcgggaattcgattAAGGGAGGCACTGA |
| | | CATTA |
| 24 | int5 − 44f | gccgcgaattcactagtgCTGCCAGCTCCATG |
| | | TGAC |
| 24A | int5 − 367f | gccgcgaattcactagtgGATTTGCCCGGTTG |
| | | GAGTC |
| 25 | int6 + 31r | ggccgcgggaattcgattAGTCACTCGCCACT |
| | | GCC |
| 26 | ABO432f | gccgcgaattcactagtgcCACCGTGTCCACT |
| | | ACTATG |
| 27 | ABO766r | ggccgcgggaattcgattTGTAGGCCTGGGAC |
| | | TGG |
| 28 | ABO723f | gccgcgaattcactagtgGGAGGCCTTCACCT |
| | | ACG |
| 29 | ABO1147r | ggccgcgggaattcgattCAGAGTTTACCCGT |
| | | TCTGC |

As indicated above, each of the primers indicated in table 4A comprises a 5′ MAPH tag (the first 18 nucleotides of the primer sequences shown in lower case) and a gene-specific sequence (shown in upper case). The present invention also provides one or more of the primers indicated in table 4A above without the 5′ MAPH tag (primer sequences represented by the sequence in uppercase only). Such primers can be used to amplify the RHD and ABO gene nucleic acids. As will be appreciated by those skilled in the art the primer sequences indicated in uppercase in table 4A can be modified by the addition of additional sequences, such as different tag sequences.

Primers according to the invention may be used with or without the MAPH tags shown above. Without the tags, the primers have the following sequences:

| Primer | | |
|---|---|---|
| no. | Primer name | Sequence (5′-3′) |
| 1 | 101F | CCATAGAGAGGCCAGCACAA |
| 2 | 198R | TGCCCCTGGAGAACCAC |
| 3 | int1F | TGACGAGTGAAACTCTATCTCGAT |
| 4 | 297R | CCACCATCCCAATACCTGAAC |
| 4A | 296R | AGAAGTGATCCAGCCACCAT |
| 5 | 303F | TCCTGGCTCTCCCTCTCT |
| 6 | 397R | GTTGTCTTTATTTTTCAAAACCCT |
| 7 | 403F | GCTCTGAACTTTCTCCAAGGACT |
| 8 | 499R | CAAACTGGGTATCGTTGCTG |
| 8A | 498R | ATTCTGCTCAGCCCAAGTAG |
| 9 | 502F | CTTTGAATTAAGCACTTCACAGA |
| 9A | 503F | TTGAATTAAGCACTTCACAGAGCA |
| 10 | 5Aluint4F | AAGGACTATCAGGCCACG |
| | (RoHar) | |
| 10A | RoHar4 | CTGAAAGGAGGGAAACGGAC |
| 10B | RoHar8 | GGGCAGTGAGCTTGATAGTAGG |
| 11 | 599R | CACCTTGCTGATCTTCCC |
| 11A | 598R | TGTGACCACCCAGCATTCTA |
| 12 | 601F | AGTAGTGAGCTGGCCCATCA |
| 13 | 697R | CTTCAGCCAAAGCAGAGGAG |
| 14 | 702F | CTGGGACCTTGTTAGAAATGCTG |
| 14A | 701F | ACAAACTCCCCGATGATGTGAGTG |
| 15 | 799R | CAAGGTAGGGGCTGGACAG |
| 15A | 798R | GAGGCTGAGAAAGGTTAAGCCA |
| 16 | 801F | CTGGAGGCTCTGAGAGGTTGAG |
| 17 | 899R | GGCAATGGTGGAAGAAAGG |
| 18 | 901F | ACTGTCGTTTTGACACACAAT |
| 19 | 998R | TGTCACCCGCATGTCAG |
| 30 | 1001F | CAAGAGATCAAGCCAAAATCAGT |
| 31 | 1097R | GTGGTACATGGCTGTATTTTATTG |
| 20 | int1 − 49f | GTGAGAGAAGGAGGGTGAG |
| 21 | int2 + 62r | ATTGGCTGCTGTGGTCA |
| 22 | int3 − 33f | CTGCTCCTAGACTAAACTTC |
| 23 | int4 + 52r | AAGGGAGGCACTGACATTA |
| 24 | int5 − 44f | CTGCCAGCTCCATGTGAC |
| 24A | int5 − 367f | GATTTGCCCGGTTGGAGTC |
| 25 | int6 + 31r | AGTCACTCGCCACTGCC |
| 26 | ABO432f | CACCGTGTCCACTACTATG |
| 27 | ABO766r | TGTAGGCCTGGGACTGG |
| 28 | ABO723f | GGAGGCCTTCACCTACG |
| 29 | ABO1147r | CAGAGTTTACCCGTTCTGC |

The primers of the present invention can be used in any method. In particular, the primer sequences may be used as probes or as primers. Preferably the primers are used in genotyping analysis, particularly blood group analysis, especially methods of RHD and/or ABO genotyping analysis.

In use, the primers are used in pairs, as indicated in the methods of the invention. The preferred pairs are as follows:

1,2;

### 3,4 or 4A;

5,6;

### 7,8 or 8A;

### 9 or 9A or 10 or 10A or 10B,11 or 11A;

12,13;

### 14 or 14A,15 or 15A;

16,17;
18,19;
20,21;
22,23;

### 24 or 24A,25;

26,27;
28,29; and
30,31

The primers may be labelled to allow easy detection.

The primers of the invention and those used in methods of the invention may be varied by the skilled addressee. For example, the lengths of the primers may be varied. This would lead to a change in Tₘ for the primers. This could then affect the annealing temperature of the PCR reaction. The length of the primers may be chosen so that the Tₘ value for a primer is under 70° C.

Substitution of bases could be made at the 5′ end of the primers without affecting the RHD specificity of the PCR reaction.

It is preferred that the AG value for primer-duplexing is less than −10 kcal/mole.

The primers according the seventh aspect of the invention and the primers used in the earlier aspects of the invention may be modified by shortening or extending the primers to include further parts of the sequence to be recognised, or by moving the primer sequence along the sequence to be recognised. Equally the primers may be modified slightly by changing one or more, preferably no more than five, more preferably no more than three, even more preferably no more than two nucleotides. Resultant primers are known as functional variants, namely variants of the original primers that are specific to the same sequences and form part of the invention.

According to an eighth aspect of the invention, there is provided a gene chip having a plurality of attached probe sequences enabling the identification of one or more of the PCR products produced by the methods indicated above. Preferably the gene chip comprises sufficient probe sequences to enable the detection of all possible PCR products produced by using the methods indicated above.

As will be appreciated by those skilled in the art the methods of the present invention may be performed in combination with any other genotyping methods. For example, the methods of genotyping the RHD and ABO genes may be combined with methods of genotyping other blood genes or any other genes. Preferably all the genotyping methods are performed using multiplex PCR. It is particularly preferred that a series of primers are used to amplify specific nucleotides sequences to be genotyped. The primers used preferably all have the same 5′ tag sequences enabling subsequent amplification of all the nucleotide sequences using primers specific to the tag sequences.

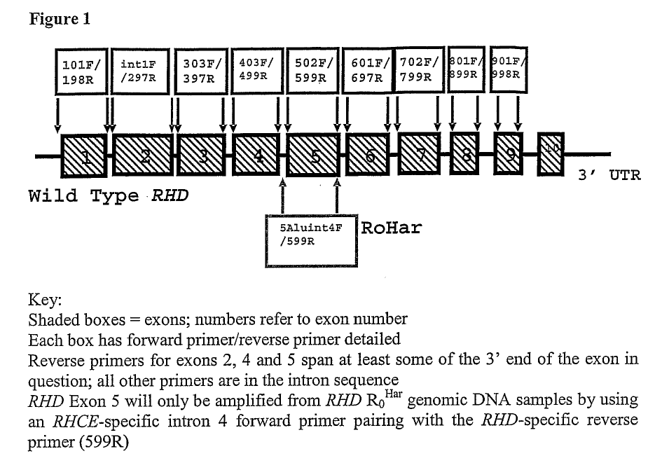

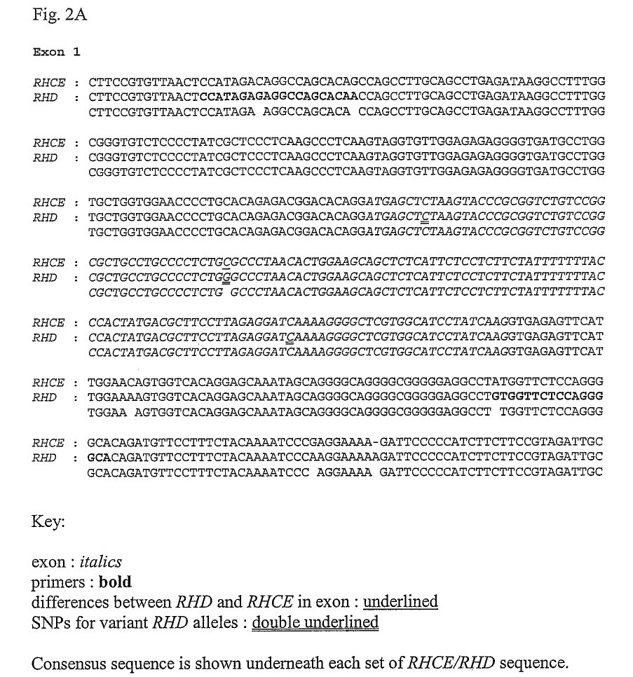

Methods and primers in accordance with the invention will now be described, by way of example only, with reference to FIGS. 1 to 11 in which:

FIG. 1 illustrates the location design of the RHD primers;

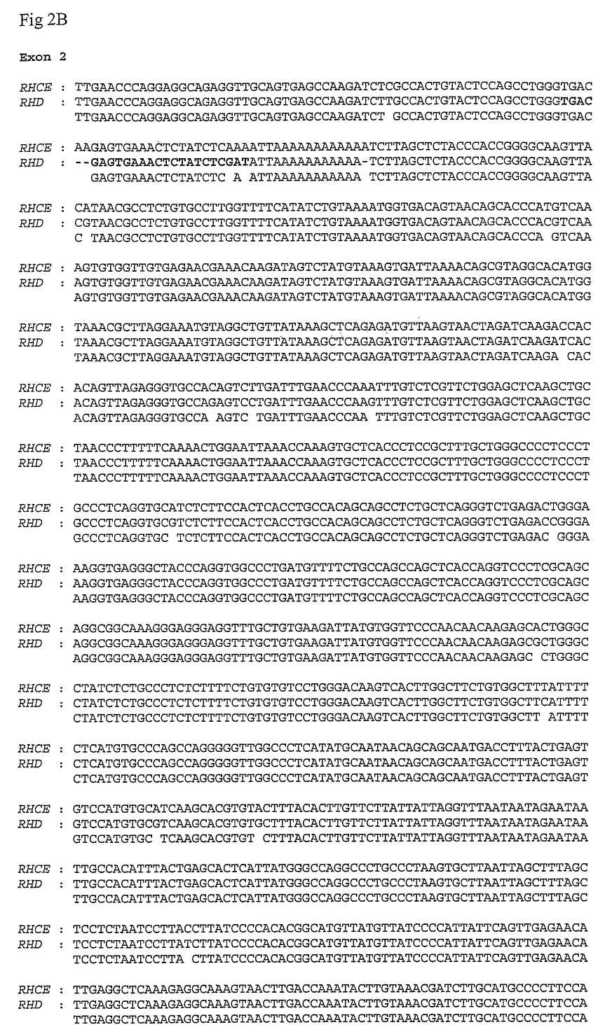

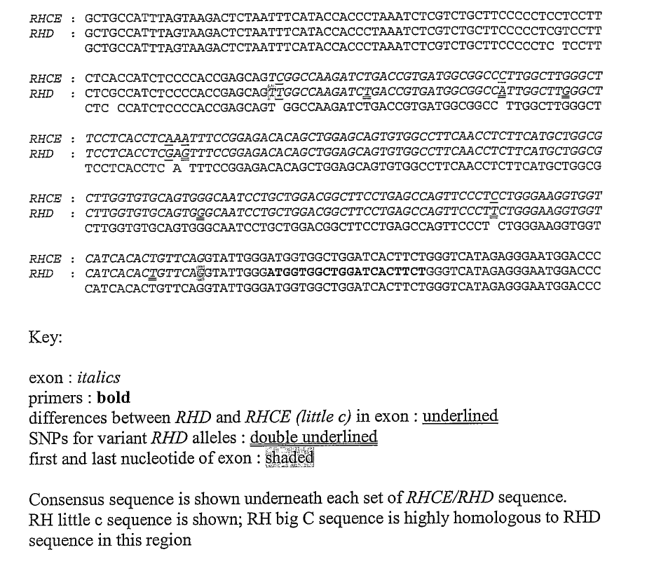

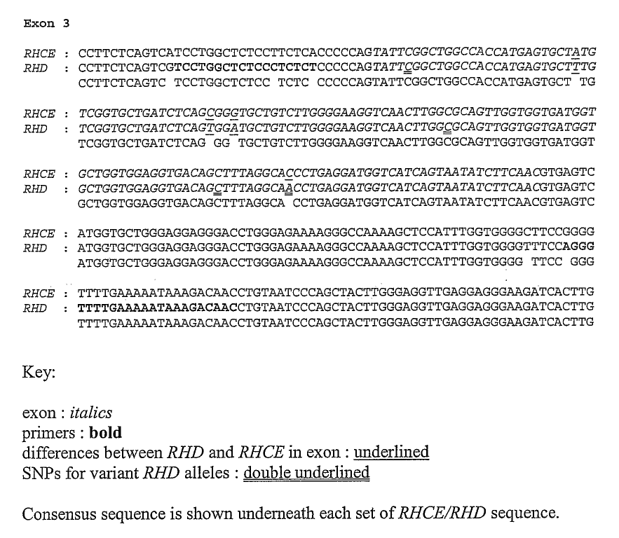

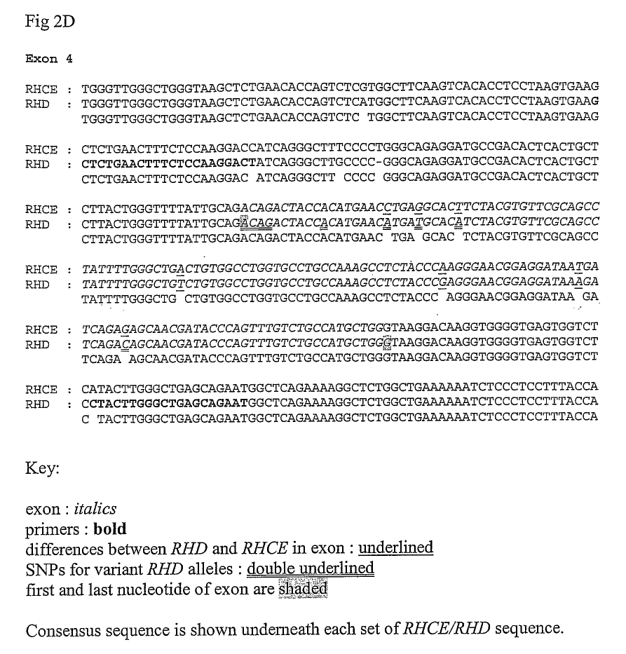

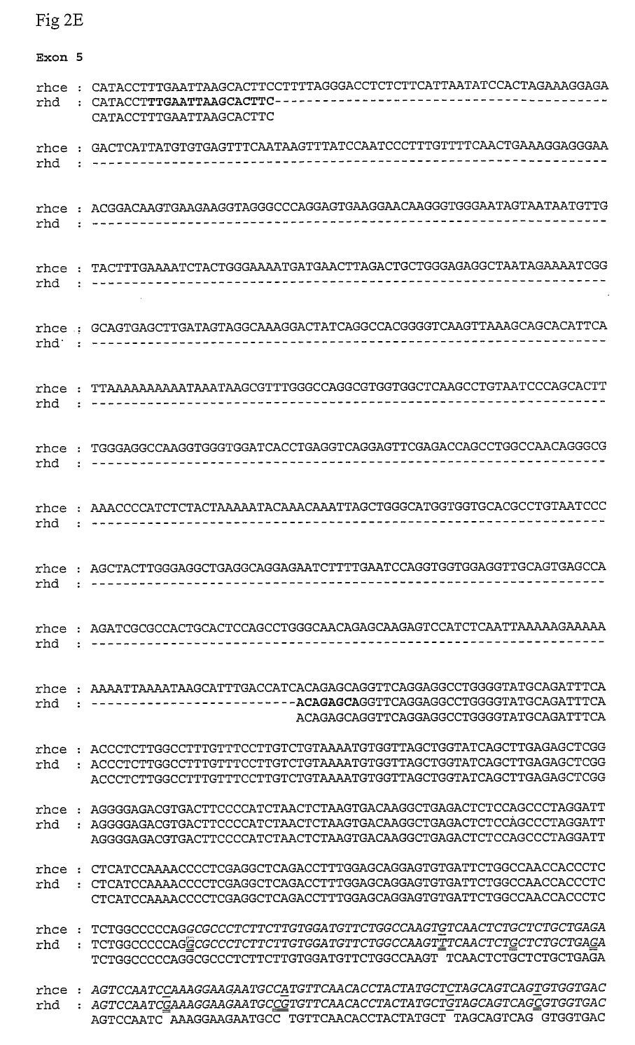

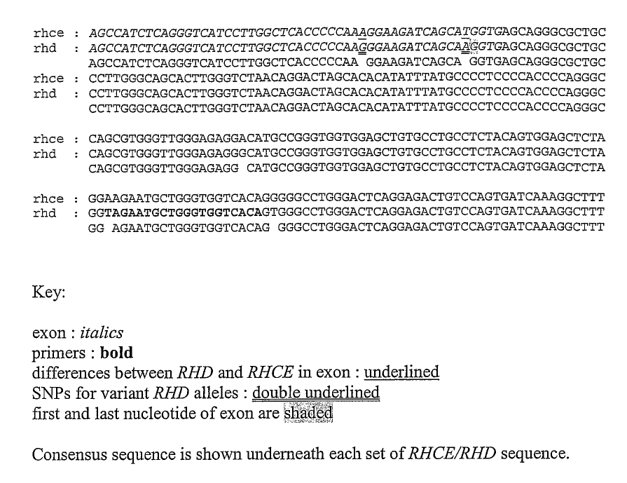

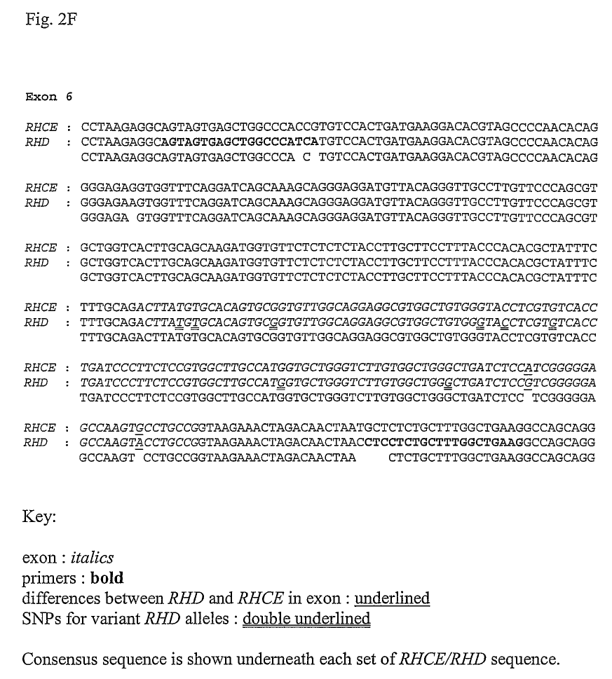

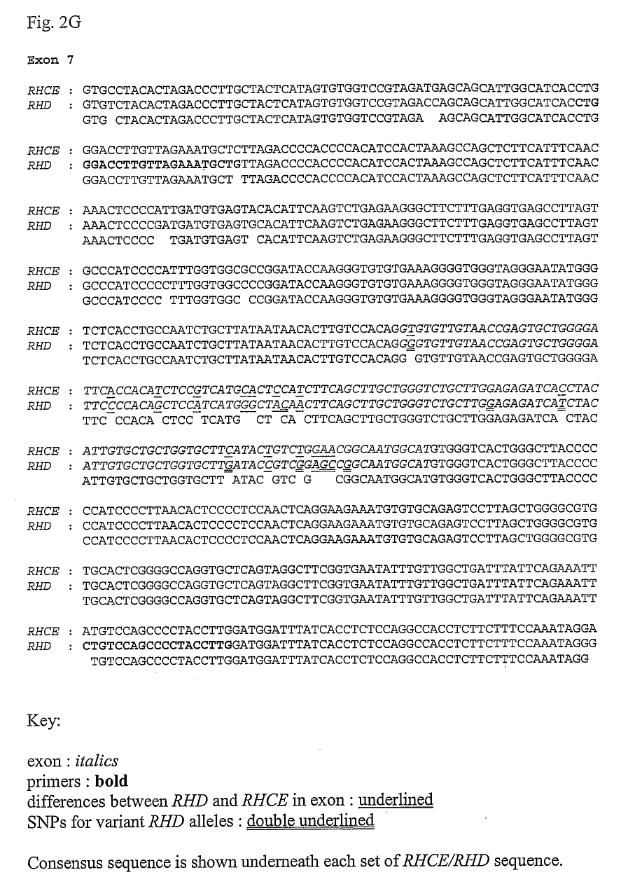

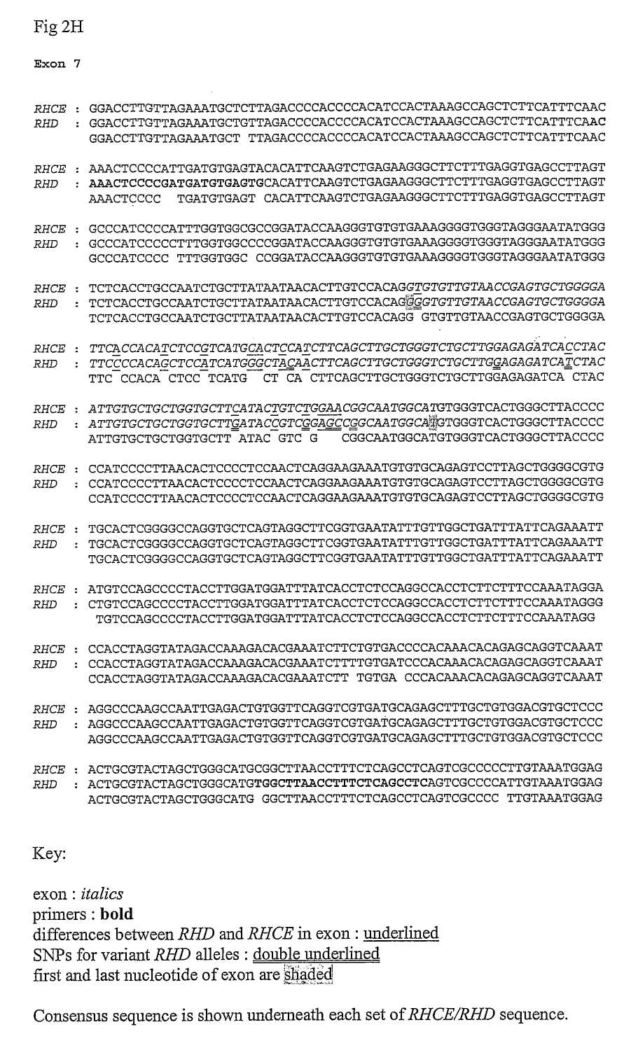

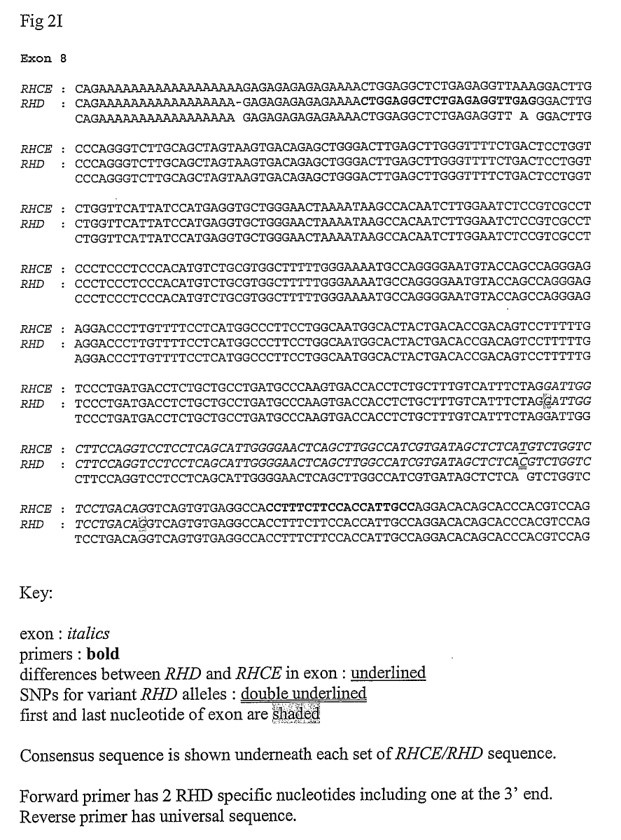

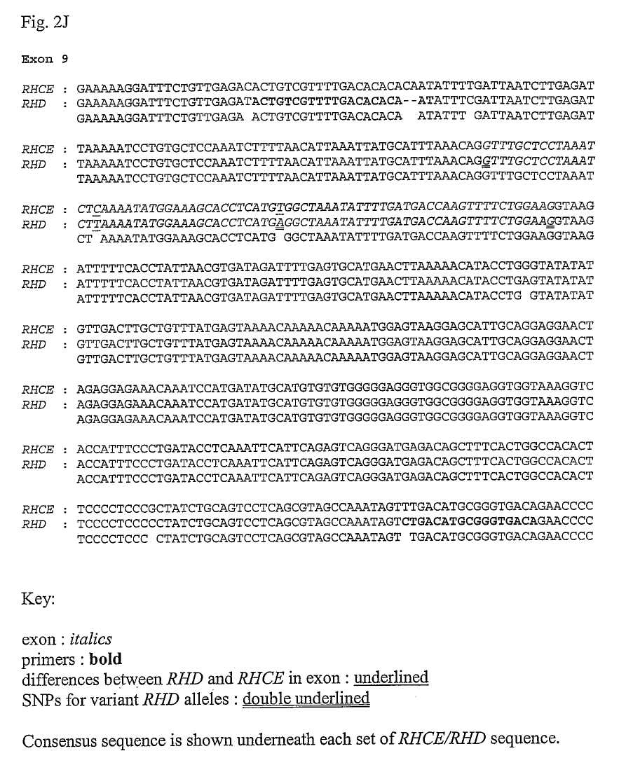

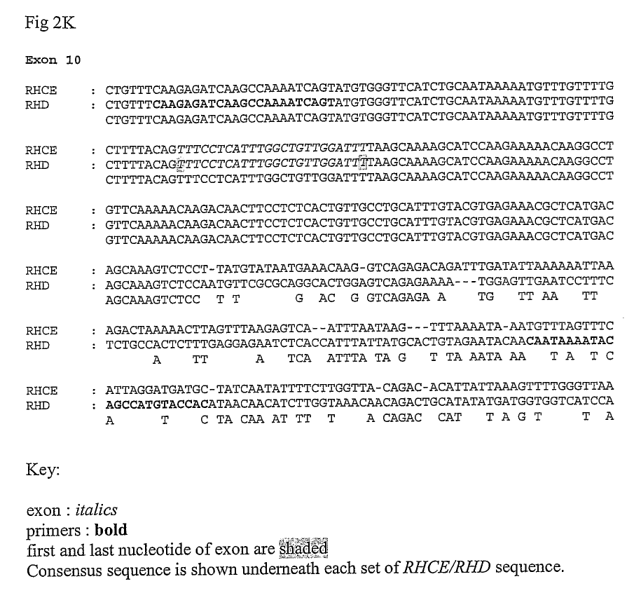

FIG. 2 illustrates RHD primers for amplification of exon 1 (FIG. 2A), exon 2 (FIG. 2B), exon 3 (FIG. 2C), exon 4 (FIG. 2D), exon 5 (FIG. 2E), exon 6 (FIG. 2F), exon 7 (FIG. 2G), exon 7 alternative primers (FIG. 2H), exon 8 (FIG. 2I) exon 9 (FIG. 2J) and exon 10 (FIG. 2K) in the RHD MPX PCR method of the invention;

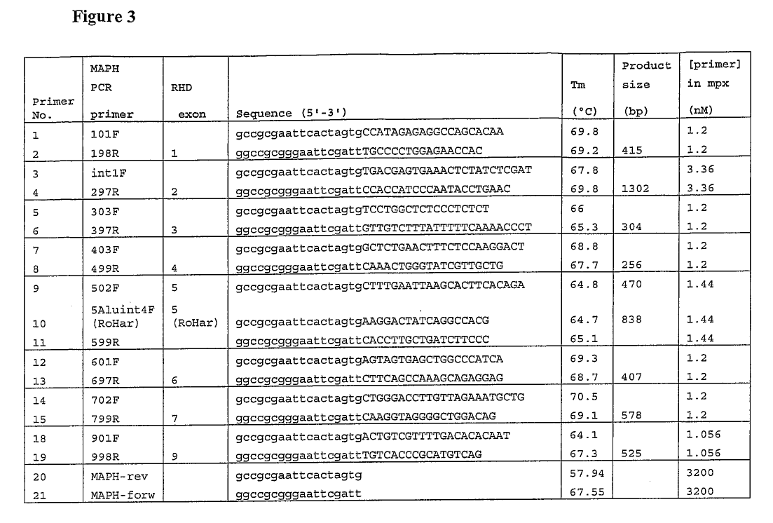

FIG. 3 shows RHD primer sequences in accordance with the invention;

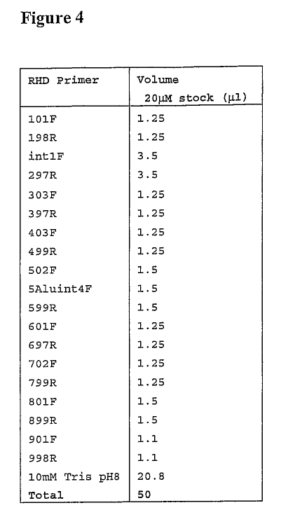

FIG. 4 shows a RHD primer mix used in a method in accordance with the invention;

FIG. 5A shows ABO primer sequences in accordance with the invention, and FIG. 5B shows the primer location in the ABO gene sequence, wherein shaded letters denote the gene-specific primer sequences, lower case letters denote intron sequence, upper case letters denote exon sequence, bold font letters denote important allele-discriminating nucleotides. The numbers indicate the nucleotide number in the ABO gene coding sequence. The A¹ allele sequence is the consensus sequence and is shown in this figure;

FIG. 6 illustrates the results of the gel electrophoresis of RHD gene amplification products from a RHD MPX PCR reaction in accordance with the invention including a primer pair for exon 8;

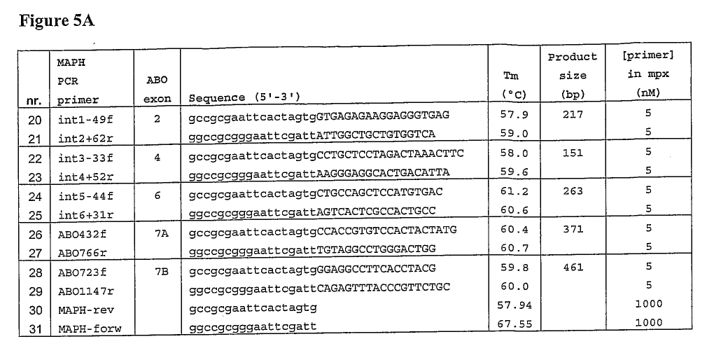

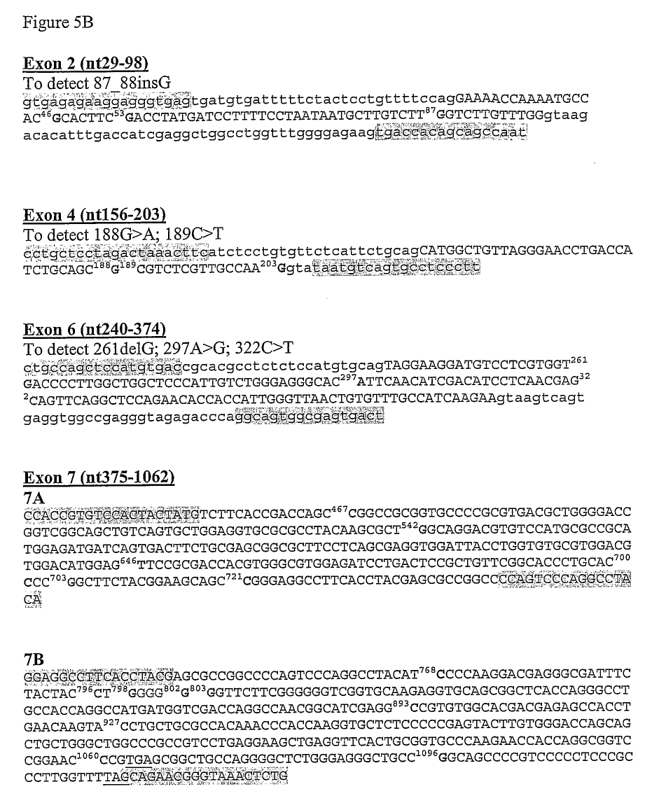

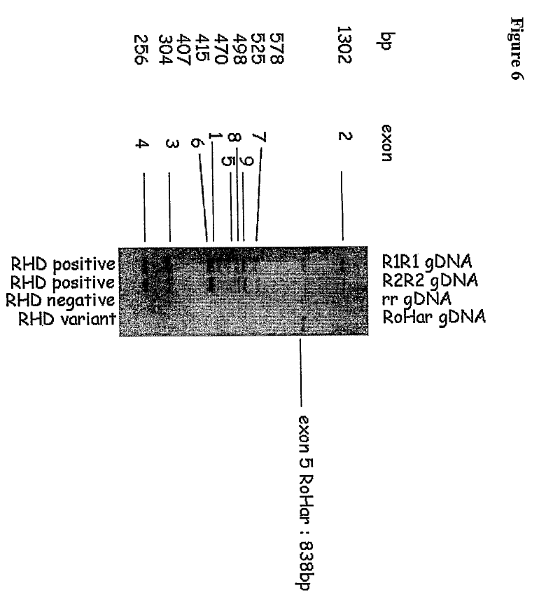

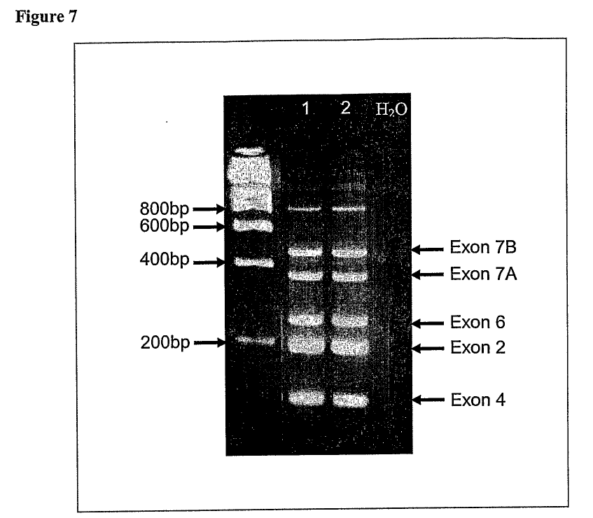

FIG. 7 illustrates the results of the gel electrophoresis of ABO gene amplification products from an ABO MPX PCR reaction in accordance with the invention;

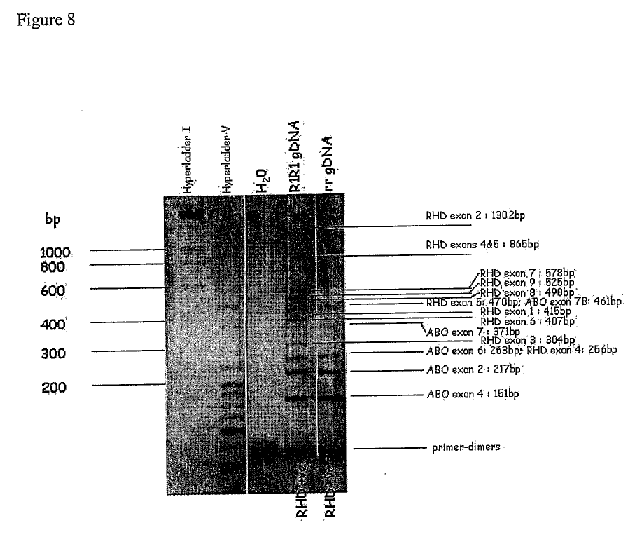

FIG. 8 illustrates the results of the gel electrophoresis of RHD and ABO gene amplification products from a RHD and ABO MPX PCR reaction in accordance with the invention including a primer pair for exon 8.

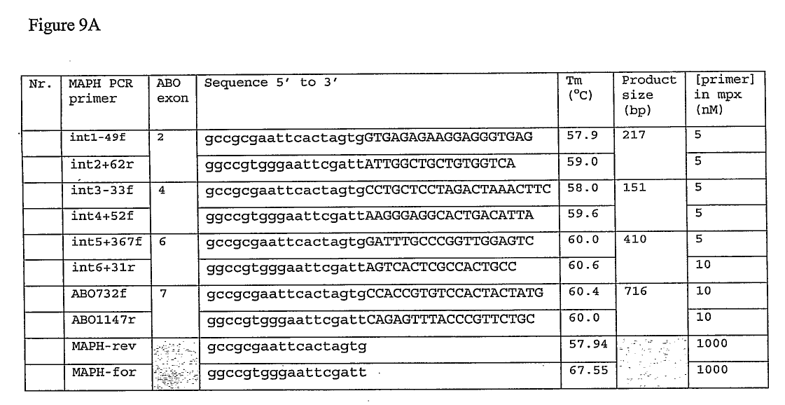

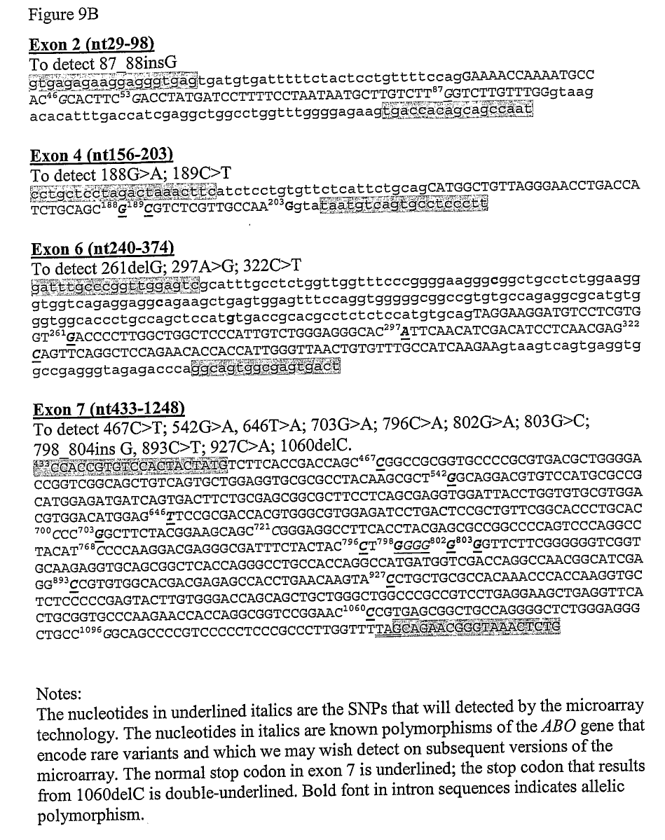

FIG. 9A shows alternative ABO primer sequences in accordance with the invention, and FIG. 9B shows the primer location in the ABO gene sequence, wherein shaded letters denote the gene-specific primer sequences, lower case letters denote intron sequence, upper case letters denote exon sequence, bold font letters denote important allele-discriminating nucleotides. The numbers indicate the nucleotide number in the ABO gene coding sequence. The A₁ allele sequence is the consensus sequence and is shown in this figure;

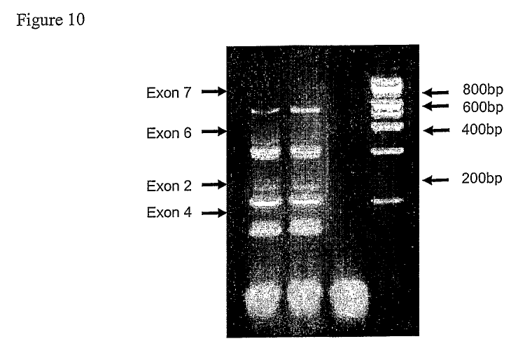

FIG. 10 illustrates the results of the gel electrophoresis of ABO gene amplification products from an ABO MPX PCR reaction in accordance with the invention; and

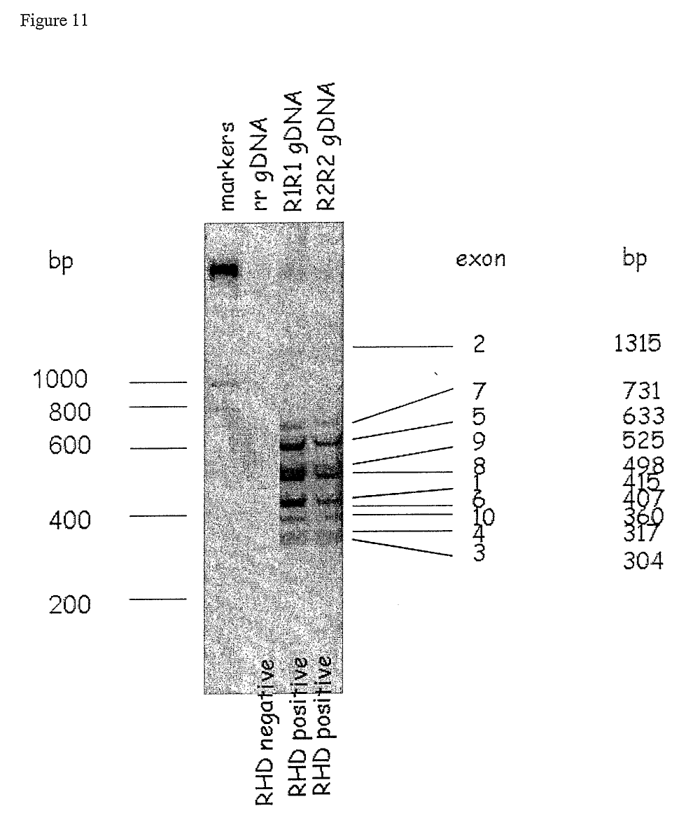

FIG. 11 illustrates the results of the gel electrophoresis of RHD gene amplification products from a RHD MPX PCR reaction in accordance with the invention including a primer pair for exon 8;

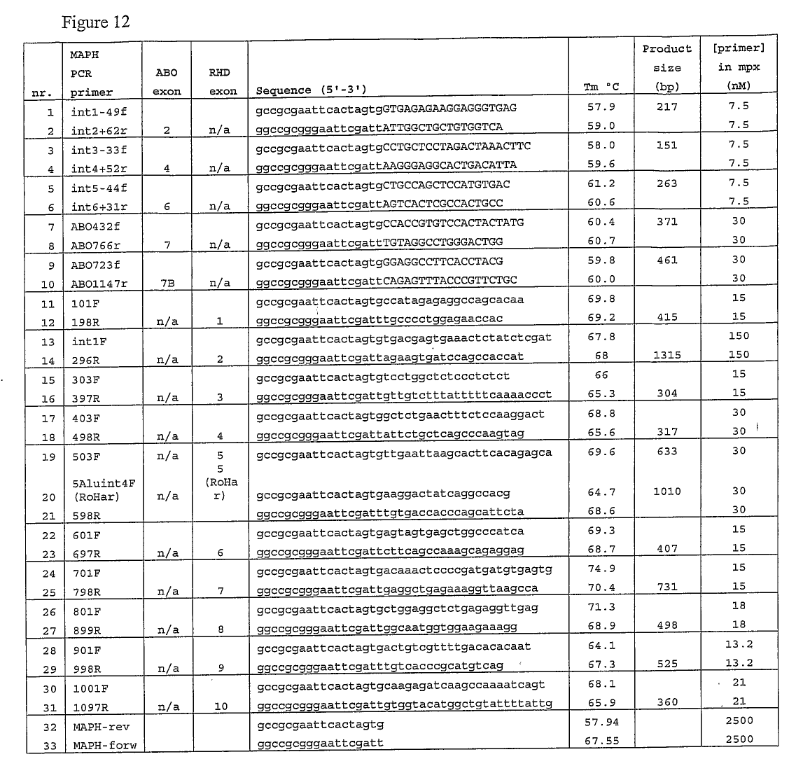

FIG. 12 shows primers according to the invention.

### EXAMPLES

### RHD Primer Design

The primers were designed or selected to ensure that the exon sequence for exons 1 to 10 inclusive of RHD is amplified by the RHD MPX PCR of the invention. The location design of the RHD primers is illustrated in FIG. 1. RHD primers are shown in FIG. 3.

The design of primers was performed using Oligo v6.0 primer design software (Molecular Biology Insights, Inc.). The Oligo v6.0 software allows a collection of primer sequences to be electronically multiplexed—this enables detection of any conflicts between the primers and checking for possible primer-dimer formations. Primers were redesigned if they were found to self-dimerize or if they were found to be incompatible with a large majority of the other primers in the multiplex. The primer sequences of a pair were chosen so that they were compatible i.e. ensuring that primer-dimer formation was limited. The lengths of the primers were chosen so that the Tₘ value for a primer was under 70° C.

Primers were also assessed using NetPrimer (PREMIER Biosoft International), a web-based program that gives each primer a rating up to 100% and also checks for primer-dimer formation. Primers were chosen for the multiplex using a combination of choosing the highest rating primers from NetPrimer results and ones which were compatible with the highest number of other primers from the Oligo v6.0 MPX results. Primers were designed to ensure that the region amplified included the known single nucleotide polymorphisms (SNPs) to be detected for the RHD gene. This generally meant that the primer positions were located in the intron sequence surrounding the exon in question. The SNP positions for the RHD gene were mapped onto the sequence data for this gene, with the RHD sequence data (introns and exons) having been aligned with the sequence data for the closely related gene RHCE. Variant RHD alleles will be detected by the MPX PCR in combination with a gene chip. An example is illustrated in FIG. 2 for RHD exons 1 to 10 primers. Primers for the RHD MPX were checked against the RHCE sequence to ensure specificity for the RHD gene.

FIG. 2A shows an alignment of RHD and RHCE sequences for exon 1 (shown in italics). The differences between the two genes in the exon are underlined. The positions of three SNPs are shown (double underlined):

| SNP | allele |
|---|---|
| C8G | weak D type 3 |
| G48A | RHD W16X (RHD negative allele) |
| C121T | RHD Q41X (RHD negative allele) |

The primer sequence positions (10F, 198R) are shown in bold (without the MAPH tags).

Similarly, FIGS. 2B-2K show the RHD and RHCE sequences, and SNPs and primers for exons 2 to 10.

The initial exon 2 forward primer was found to amplify from RHC as well as RHD so the primer sequence was changed to the one disclosed in Legler, T J et al., Transfusion Medicine 2001 11, 383-388). A total of 10 different primers were tried for exon 2 in order to achieve RHD specificity. Six primers were tried for exon 2 where base changes have been introduced into the sequence. These were tested because they would have amplified a smaller product for exon 2 but the sequence changes did not result in RHD specificity.

The majority of the primers have 3′ RHD specific ends but two of the primers are complementary to RHD and RHCE sequence (exon 2 reverse and exon 8 reverse). Exon 5 forward primer spans a region of sequence where there is an insert in RHCE but not in RHD.

For exons 4 and 5, previously published reverse primer sequences could be used (Maaskant-van Wijk et al., Transfusion, 38, 1015-1021, 1998).

### ABO Primer Design

ABO primers are shown in FIG. 5A. Primers were designed to amplify exons 2, 4, 6 and 7 of the ABO gene. In one design, for optimal amplification in a multiplex reaction, PCR products of 400 bp or less were desired and consequently, primers were selected to amplify exon 7 in two parts: 7A and 7B. Fragment 7B is 461 base pairs long but is readily amplified under the conditions described and is required to incorporate all known allele variants within this DNA sequence. The primer pairs were designed to be inclusive of all known mutations in the exon and were placed in non-variable regions of the introns. Allele-determining mutations are denoted in bold font in FIG. 5B and their position in the coding sequence of the gene denoted by the nucleotide number given in superscript. Subsequent to the initial design, an intron 5 polymorphism was found in primer int5-44F. Other intron 5 gene specific primers were identified and int5-367F was substituted into the assay (see FIGS. 9A and 9B). The intent of the microarray is that allele-specificity is determined by specific oligonucleotide probes that will bind to gene-specific PCR products, and that was our goal for ABO-specific, exon-specific primer selection.

Primer sequences were designed de novo. All primer pairs were checked using the Oligo v6.0 primer design software to evaluate melting temperatures, possible primer-dimer formation and hairpin formation. The length of the primers was selected to give a melting temperature of ˜60° C. The sequences of the primers are shown in the following table:

| | MAPH | | |
|---|---|---|---|
| | PCR | ABO | |
| nr. | primer | exon | Sequence (5′-3′) |
| 20 | int1 − 49f | 2 | gccgcgaattcactagtgGTGAGAGAAGGAG |
| | | | GGTGAG |
| 21 | int2 + 62r | | ggccgcgggaattcgattATTGGCTGCTGTG |
| | | | GTCA |
| 22 | int3 − 33f | 4 | gccgcgaattcactagtgCCTGCTCCTAGAC |
| | | | TAAACTTC |
| 23 | int4 + 52r | | ggccgcgggaattcgattAAGGGAGGCACTG |
| | | | ACATTA |
| 24 | int5 − 44f | 5 | gccgcgaattcactagtgCTGCCAGCTCCAT |
| | | | GTGAC |
| 25 | int6 + 31r | | ggccgcgggaattcgattAGTCACTCGCCAC |
| | | | TGCC |
| 26 | ABO432f | 7A | gccgcgaattcactagtgCCACCGTGTCCAC |
| | | | TACTATG |
| 27 | ABO766r | | ggccgcgggaattcgattTGTAGGCCTGGGA |
| | | | CTGG |
| 28 | ABO723f | 7B | gccgcgaattcactagtgGGAGGCCTTCACC |
| | | | TACG |
| 29 | ABO1147r | | ggccgcgggaattcgattCAGAGTTTACCCG |
| | | | TTCTGC |

Multiplex primer details for ABO-specific amplification. Lower case letters denote the MAPH tag sequence. Upper case letters denote the gene-specific sequence.

### Multiplex PCR Blood RHD Gene Analysis

Genomic DNA was isolated from adult peripheral blood using the QIAamp DNA Blood Mini kit (Qiagen Ltd.). The amount of genomic DNA in each sample was quantitated by measuring the absorbance at 260 nm. Standard genomic DNA samples were used to assess the reliability of the multiplex PCR:

### R1R1=CDe/CDe

R2R2=cDE/cDE
rr=cde/cde
r′r=Cde/cde
r″r=cdE/cde
R0r=cDe/cde

A 25 μl PCR mix consisted of:

per 25 μl MPX reaction

| | | |
|---|---|---|
| 12.5 | μl | 2x Mastermix ^{#} |
| 0.06 | μl | RHD primer mix |
| 0.8 | μl | 100 μM MAPH forward |
| 0.8 | μl | 100 μM MAPH reverse |
| 0.25 | μl | Mg²⁺ (50 mM, Bioline) |
| 9.59 | μl | H₂O |
| 1 | μl | 100 ng/μl DNA |
| 25 | μl | Total |

#2×Mastermix=Qiagen multiplex PCR buffer which comprises all the necessary components for performing the PCR reaction, including HotStarTaq DNA Polymerase, Mg²⁺ and necessary dNTPs.

Primers were supplied by Operon Biotechnologies (formerly Qiagen). A suitable primer mix is shown in FIG. 4. The primer mix shown in FIG. 4 is a guide and variations may be made to the primer mix to change the ratio of the various primer pairs used.

Multiplex amplification and probe hybridization (MAPH)-tagged PCR primers are used to multiplex amplify gene fragments by producing “hybrid” PCR primers that have a 5′ end MAPH tag and a 3′ gene specific fragment. In the initial stages of the PCR the gene fragments will be amplified by these hybrid primers. Included in the PCR mix are MAPH forward and reverse primers that will amplify every PCR product amplified by the hybrid primers. This provides the multiplex reaction with uniformity and up to 20 gene fragments can be amplified in this manner. A modification of MAPH is disclosed by White et al (White, S et al Am. J. Hum. Genet. 2002 August; 71(2):365-74) including the flanking sequences, which are referred to as “MAPH forward” and “MAPH reverse” (FIG. 3). The flanking sequences were supplied by Sanquin.

The amplification protocol was:

Multiplex PCR programme

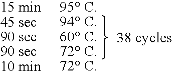

This was an adaptation of the protocol detailed by Qiagen for the Multiplex PCR buffer kit. The denaturation time has been extended, the annealing temperature chosen is in the middle of the range given (57-63° C.) and the number of cycles is in the middle of the range given (30-45 cycles).

DHAR genomic DNA samples will have intron 4 of RHCE rather than intron 4 of RHD. Due to the location of the forward primer for exon 5, no exon 5 product would be amplified for DHAR samples with the original set of MPX primers. Therefore we have designed a forward primer 5′ of the Alu sequence in intron 4 in a region that is RHCE specific. This primer is compatible with the reverse primer for exon 5 (RHD-specific).

### Multiplex PCR Blood ABO Gene Analysis

Genomic DNA was isolated from adult peripheral blood by either the QIAamp DNA Blood Mini Kit (Qiagen Ltd.) or by a modified salting-out procedure (Miller et al (1988) Nuc. Ac. Res. 16 1215). DNA concentration was determined spectrophotometrically at 260 nm, and diluted to 100 ng/μL. Samples of different common ABO blood groups were selected for amplification.

per 25 μl MPX reaction

| | | |
|---|---|---|
| 12.5 | μl | 2x Mastermix ^{#} |
| 0.25 | μl | ABO primer mix (0.5 μM) |
| 0.5 | μl | 50 μM MAPH forward |
| 0.5 | μl | 50 μM MAPH reverse |
| 10.25 | μl | H₂0 |
| 1 | μl | 100 ng/μl DNA |
| 25 | μl | Total |

# 2×Mastermix=Qiagen multiplex PCR buffer which comprises all the necessary components for performing the PCR reaction, including HotStarTaq DNA Polymerase, Mg²⁺ and necessary dNTPs.

The ABO primer mix comprises:

| | Volume |
|---|---|
| ABO Primer | 10 μM stock (μl) |
| int1 − 49f | 2 |
| int2 + 62r | 2 |
| int3 − 33f | 2 |
| int4 + 52r | 2 |
| int5 − 44f | 2 |
| int6 + 31r | 2 |
| ABO432f | 2 |
| ABO766r | 2 |
| ABO723f | 2 |
| ABO1147r | 2 |
| 10 mM Tris pH 8 | 20 |
| Total | 40 |

Amplification was performed in 0.2 mL PCR tubes in either a PE 9700 or a PE 2700 thermal cycler (Perkin Elmer/Cetus, Norwalk, Conn.) under the following conditions:

### Multiplex PCR Programme

Multiplex PCR programme

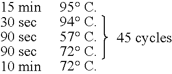

Amplified products were assessed by running 10 μL of each reaction on either a 3% agarose gel (prepared in house) or a 5-20% polyacrylamide gel (Novex Gels, Invitrogen, Inc.). A representative gel is shown in FIG. 7 and shows the robust nature of the amplification reaction. Faint bands of 700 bp and higher indicate the low levels of amplification of larger gene-specific fragments as predicted.

In an alternative example, the following mixes were used:

per 25 ul MPX reaction

| | | |
|---|---|---|
| 12.5 | uL | 2x Mastermix |
| 0.25 | uL | ABO primer mix |
| 0.4 | uL | 50 uM MAPH forward |
| 0.4 | uL | 50 uM MAPH reverse |
| 10.45 | uL | H₂O |
| 1 | uL | 100 ng/uL DNA |
| 25 | uL | Total |

Stock ABO primer mix used in the reaction above was prepared as follows:

| ABO primer | Volume 10 uM stock (uL) |
|---|---|
| int1 − 49f | 2.5 |
| int2 + 62r | 2.5 |
| int3 − 33f | 2.5 |
| int4 + 52r | 2.5 |
| int5 + 367f | 2.5 |
| int6 + 31r | 5 |
| ABO432f | 5 |
| ABO1147r | 5 |
| 10 mM Tris pH 8 | 72.5 |
| Total | 100 |

The primers used in this example, the regions amplified and the resulting gel are shown in FIGS. 9A, 9B and 10.

### Multiplex PCR Blood RHD and ABO Gene Analysis

Genomic DNA was isolated and quantified as before. The primer mixes used were as indicated for the individual RHD MPX PCR and the ABO MPX PCR. However, final concentrations of primers in the reaction were different to those detailed above due to the reaction mix setup below.

A 25 μl PCR mix consisted of:

per 25 μl MPX reaction

| | | |
|---|---|---|
| 12.5 | μl | 2x Mastermix ^{#} |
| 0.085 | μl | RHD primer mix |
| 0.2 | μl | ABO primer mix |
| 1.3 | μl | 100 μM MAPH forward |
| 1.3 | μl | 100 μM MAPH reverse |
| 0.6 | μl | Mg²⁺ (50 mM, Bioline) |
| 8.015 | μl | H₂0 |
| 1 | μl | 100 ng/μl DNA |
| 25 | μl | Total |

#2×Mastermix=Qiagen multiplex PCR buffer which comprises all the necessary components for performing the PCR reaction, including HotStarTaq DNA Polymerase, Mg²⁺ and necessary dNTPs.

The PCR amplification reactions were performed as indicated above, except that the following programme was used:

Multiplex PCR programme

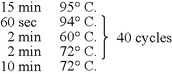

Amplified products were assessed as indicated above. A representative gel is shown in FIG. 8 and shows the robust nature of the amplification reaction.

In an alternative example, the following mixes were used:

ABO and RHD primer

mix

| | Volume |
|---|---|
| ABO Primer | 10 uM stock (ul) |
| int1 − 49f | 1.25 |
| int2 + 62r | 1.25 |
| int3 − 33f | 1.25 |
| int4 + 52r | 1.25 |
| int5 − 44f | 1.25 |
| int6 + 31r | 1.25 |
| ABO432f | 5 |
| ABO766r | 5 |
| ABO723f | 5 |
| ABO1147r | 5 |

| | Volume |
|---|---|
| RHD Primer | 20 uM stock (ul) |
| 101F | 1.25 |
| 198R | 1.25 |
| int1F | 12.5 |
| 296R | 12.5 |
| 303F | 1.25 |
| 397R | 1.25 |
| 403F | 2.5 |
| 498R | 2.5 |
| 503F | 2.5 |
| 598R | 2.5 |
| 5Aluint4F | 2.5 |
| 601F | 1.25 |
| 697R | 1.25 |
| 701F | 1.25 |
| 798R | 1.25 |
| 801F | 1.5 |
| 899R | 1.5 |
| 901F | 1.1 |
| 998R | 1.1 |
| 1001F | 1.75 |
| 1097R | 1.75 |
| 10 mM Tris pH 8 | 16.3 |
| Total | 100 |

per 25 ul mpx reaction

| | |
|---|---|
| 12.5 ul | 2x Mastermix^{#} |
| 1.5 ul | ABO/RHD primer mix |
| 0.625 ul | 100 mM MAPH forw |
| 0.625 ul | 100 uM MAPH rev |
| 8.75 ul | H₂O |
| 1 ul | 100 ng/ul DNA |
| 25 ul | Total |

^{#}2x Mastermix = Qiagen multiplex PCR buffer
NOTE MALPH volumes will vary according to stock concentration and
depending on whether the primers are added from one combined MAPH
mix
NOTE DNA has been used at 100 ng/ul but volumes could be adjusted to
use at 40 ng/ul
Multiplex PCR programme

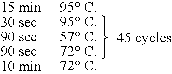

### Multiplex PCR Results

The MPX PCR amplifies all the products required. These products are visible by gel electrophoresis as shown in FIG. 6 (RHD gene amplification products) and FIG. 7 (ABO gene amplification products). Alternatively, the products are visible by GeneScan® analysis software (Applied Biosystems) using a capillary microsequencer (Applied Biosystems). The products have also been sequenced to ensure that the correct amplicons are being amplified.

The size of each amplicon and the RHD exon from which it is derived are indicated on the left of FIG. 6. In FIG. 6 “gDNA” means genomic DNA. A product specific for exon 5 of the RHD R₀^{Har} gene variant (DHAR) is also highlighted. This product is not obtained from normal D-positive and D-negative samples. Primer pairs were also tested individually to ensure RHD specificity.

In FIG. 7, the ABO exon from which each amplicon is derived is indicated on the right of the figure. Exon 4 is 151 bp; exon 2 is 217 bp; exon 6 is 263 bp; exon 7A is 371 bp; and exon 7B is 461 bp. The numbers on the left of the figure indicate the size of the DNA marker bands.

In FIG. 8, the RHD and ABO exon from which each amplicon is derived is indicated on the right of the figure. The numbers on the left of the figure indicate the size of the DNA marker bands.

The amplified nucleic acids may then be hybridized to further sequences in an array such as gene chip.

Although conditions for MPX PCR are described herein, those skilled in the art will be aware that any appropriate MPX PCR conditions may be used.

1. A method of RHD genotyping analysis, by multiplex PCR, the method comprising contacting RHD gene nucleic acids from a subject with the one or more of the following primer pairs 1,2;3,4 or 4A;5,6;7,8 or 8A;9 or 9A or 10 or 10A or 10B, 11 or 11A;12,13;14 or 14A,15 or 15A;16,17;18,19; and 30,31 from the following table, wherein the primer pairs may comprise the entire sequence shown in the table or the sequence shown in uppercase:

| | | | |
|---|---|---|---|
| | | | SEQ |
| Primer | Primer | | ID |
| no. | name | Sequence (5′-3′) | NO |
| | | | |
|---|---|---|---|
| 1 | 101F | gccgcgaattcactagtgCCATAGAGAGGCC | 1 |
| | | AGCACAA | |
| 2 | 198R | ggccgcgggaattcgattTGCCCCTGGAGAA | 2 |
| | | CCAC | |
| 3 | int1F | gccgcgaattcactagtgTGACGAGTGAAAC | 3 |
| | | TCTATCTCGAT | |
| 4 | 297R | ggccgcgggaattcgattCCACCATCCCAAT | 4 |
| | | ACCTGAAC | |
| 4A | 296R | ggccgcgggaattcgattAGAAGTGATCCAG | 5 |
| | | CCACCAT | |
| 5 | 303F | gccgcgaattcactagtgTCCTGGCTCTCCC | 6 |
| | | TCTCT | |
| 6 | 397R | ggccgcgggaattcgattGTTGTCTTTATTT | 7 |
| | | TTCAAAACCCT | |
| 7 | 403F | gccgcgaattcactagtgGCTCTGAACTTTC | 8 |
| | | TCCAAGGACT | |
| 8 | 499R | ggccgcgggaattcgattCAAACTGGGTATC | 9 |
| | | GTTGCTG | |
| 8A | 498R | ggccgcgggaattcgattATTCTGCTCAGCC | 10 |
| | | CAAGTAG | |
| 9 | 502F | gccgcgaattcactagtgCTTTGAATTAAGC | 11 |
| | | ACTTCACAGA | |
| 9A | 503F | gccgcgaattcactagtgTTGAATTAAGCAC | 12 |
| | | TTCACAGAGCA | |
| 10 | 5Aluint4F | gccgcgaattcactagtgAAGGACTATCAGG | 13 |
| | (RoHar) | CCACG | |
| 10A | RoHar4 | gccgcgaattcactagtgCTGAAAGGAGCGA | 14 |
| | | AACGGAC | |
| 10B | RoHar8 | gccgcgaattcactagtgGGGCAGTGAGCTT | 15 |
| | | GATAGTAGG | |
| 11 | 599R | ggccgcgggaattcgattCACCTTGCTGATC | 16 |
| | | TTCCC | |
| 11A | 598R | ggccgcgggaattcgattTGTGACCACCCAG | 17 |
| | | CATTCTA | |
| 12 | 601F | gccgcgaattcactagtgAGTAGTGAGCTGG | 18 |
| | | CCCATCA | |
| 13 | 697R | ggccgcgggaattcgattCTTCAGCCAAAGC | 19 |
| | | AGAGGAG | |
| 14 | 702F | gccgcgaattcactagtgCTGGGACCTTGTT | 20 |
| | | AGAAATGCTG | |
| 14A | 701F | gccgcgaattcactagtgACAAACTCCCCGA | 21 |
| | | TGATGTGAGTG | |
| 15 | 799R | ggccgcgggaattcgattCAAGGTAGGGGCT | 22 |
| | | GGACAG | |
| 15A | 798R | ggccgcgggaattcgattGAGGCTGAGAAAG | 23 |
| | | GTTAAGCCA | |
| 16 | 801F | gccgcgaattcactagtgCTGGAGGCTCTGA | 24 |
| | | GAGGTTGAG | |
| 17 | 899R | ggccgcgggaattcgattGGCAATGGTGGAA | 25 |
| | | GAAAGG | |
| 18 | 901F | gccgcgaattcactagtgACTGTCGTTTTGA | 26 |
| | | CACACAAT | |
| 19 | 998R | ggccgcgggaattcgattTGTCACCCGCATG | 27 |
| | | TCAG | |
| 30 | 1001F | gccgcgaattcactagtgCAAGAGATCAAGC | 28 |
| | | CAAAATCAGT | |
| 31 | 1097R | ggccgcgggaattcgattGTGGTACATGGCT | 29 |
| | | GTATTTTATTG | |
| 32 | MAPH-rev | gccgcgaattcactagtg | 30 |
| 33 | MAPH-forw | ggccgcgggaattcgatt | 31 |

and amplifying the RHD gene nucleic acids.

2.-9. (canceled)

10. A method of RHD genotyping analysis, by Multiplex PCR, the method comprising contacting RHD gene nucleic acids from a subject with at least one primer selected from the following table, wherein the primer may comprise the entire sequence shown in the table or the sequence shown in uppercase:

| | | | |
|---|---|---|---|
| | | | SEQ |
| Primer | Primer | | ID |
| no. | name | Sequence (5′-3′) | NO: |
| | | | |
|---|---|---|---|
| 1 | 101F | gccgcgaattcactagtgCCATAGAGAGGCC | 1 |
| | | AGCACAA | |
| 4 | 297R | ggccgcgggaattcgattCCACCATCCCAAT | 4 |
| | | ACCTGAAC | |
| 4A | 296R | ggccgcgggaattcgattAGAAGTGATCCAG | 5 |
| | | CCACCAT | |
| 5 | 303F | gccgcgaattcactagtgTCCTGGCTCTCCC | 6 |
| | | TCTCT | |
| 6 | 397R | ggccgcgggaattcgattGTTGTCTTTATTT | 7 |
| | | TTCAAAACCCT | |
| 7 | 403F | gccgcgaattcactagtgGCTCTGAACTTTC | 8 |
| | | TCCAAGGACT | |
| 8A | 498R | ggccgcgggaattcgattATTCTGCTCAGCC | 10 |
| | | CAAGTAG | |
| 9 | 502F | gccgcgaattcactagtgCTTTGAATTAAGC | 11 |
| | | ACTTCACAGA | |
| 9A | 503F | gccgcgaattcactagtgTTGAATTAAGCAC | 12 |
| | | TTCACAGAGCA | |
| 10 | 5Aluint4F | gccgcgaattcactagtgAAGGACTATCAGG | 13 |
| | (RoHar) | CCACG | |
| 10A | RoHar4 | gccgcgaattcactagtgCTGAAAGGAGGGA | 14 |
| | | AACGGAC | |
| 10B | RoHar8 | gccgcgaattcactagtgGGGCAGTGAGCTT | 15 |
| | | GATAGTAGG | |
| 11 | 599R | ggccgcgggaattcgattCACCTTGCTGATC | 16 |
| | | TTCCC | |
| 11A | 598R | ggccgcgggaattcgattTGTGACCACCCAG | 17 |
| | | CATTCTA | |
| 12 | 601F | gccgcgaattcactagtgAGTAGTGAGCTGG | 18 |
| | | CCCATCA | |
| 13 | 697R | ggccgcgggaattcgattCTTCAGCCAAAGC | 19 |
| | | AGAGGAG | |
| 14 | 702F | gccgcgaattcactagtgCTGGGACCTTGTT | 20 |
| | | AGAAATGCTG | |
| 14A | 701F | gccgcgaattcactagtgACAAACTCCCCGA | 21 |
| | | TGATGTGAGTG | |
| 15 | 799R | ggccgcgggaattcgattCAAGGTAGGGGCT | 22 |
| | | GGACAG | |
| 15A | 798R | ggccgcgggaattcgattGAGGCTGAGAAAG | 23 |
| | | GTTAAGCCA | |
| 17 | 899R | ggccgcgggaattcgattGGCAATGGTGGAA | 25 |
| | | GAAAGG | |
| 18 | 901F | gccgcgaattcactagtgACTGTCGTTTTGA | 26 |
| | | CACACAAT | |
| 19 | 998R | ggccgcgggaattcgattTGTCACCCGCATG | 27 |
| | | TCAG | |
| 31 | 1097R | ggccgcgggaattcgattGTGGTACATGGCT | 29 |
| | | GTATTTTATTG | |

and amplifying the RHD gene nucleic acids.

11.-13. (canceled)

14. A method of ABO genotyping analysis, by multiplex PCR, the method comprising contacting ABO gene nucleic acids from a subject with one or more of the following primer pairs 20,21; 22,23; 24 or 24A,25; 26,27 and 28,29 from the following table, wherein the primer pairs may comprise the entire sequence shown in the table or the sequence shown in uppercase:

| | | | SEQ |
|---|---|---|---|
| Primer | Primer | | ID |
| no. | name | Sequence (5′-3′) | NO: |
| 20 | int1 − 49f | gccgcgaattcactagtgGTGAGAGAAG | 32 |
| | | GAGGGTGAG | |
| 21 | int2 + 62r | ggccgcgggaattcgattATTGGCTGCT | 33 |
| | | GTGGTCA | |
| 22 | int3 − 33f | gccgcgaattcactagtgcCTGCTCCTA | 34 |
| | | GACTAAACTTC | |
| 23 | int4 + 52r | ggccgcgggaattcgattAAGGGAGGCA | 35 |
| | | CTGACATTA | |
| 24 | int5 − 44f | gccgcgaattcactagtgCTGCCAGCTC | 36 |
| | | CATGTGAC | |
| 24A | int5 − 367f | gccgcgaattcactagtgGATTTGCCCG | 37 |
| | | GTTGGAGTC | |
| 25 | int6 + 31r | ggccgcgggaattcgattAGTCACTCGC | 38 |
| | | CACTGCC | |
| 26 | ABO432f | gccgcgaattcactagtgcCACCGTGTC | 39 |
| | | CACTACTATG | |
| 27 | ABO766r | ggccgcgggaattcgattTGTAGGCCTG | 40 |
| | | GGACTGG | |
| 28 | ABO723f | gccgcgaattcactagtgGGAGGCCTTC | 41 |
| | | ACCTACG | |
| 29 | ABO1147r | ggccgcgggaattcgattCAGAGTTTAC | 42 |
| | | CCGTTCTGC | |

and amplifying the ABO gene nucleic acids.

15.-18. (canceled)

19. A method of ABO genotyping analysis, by multiplex PCR, the method comprising contacting ABO nucleic acid from a subject with at least one primer selected from the following table, wherein the primer may comprise the entire sequence shown in the table or the sequence shown in uppercase:

| | | | SEQ |
|---|---|---|---|
| Primer | Primer | | ID |
| no. | name | Sequence (5′-3′) | NO: |
| 20 | int1 − 49f | gccgcgaattcactagtgGTGAGAGAAG | 32 |
| | | GAGGGTGAG | |
| 21 | int2 + 62r | ggccgcgggaattcgattATTGGCTGCT | 33 |
| | | GTGGTCA | |
| 22 | int3 − 33f | gccgcgaattcactagtgcCTGCTCCTA | 34 |
| | | GACTAAACTTC | |
| 23 | int4 + 52r | ggccgcgggaattcgattAAGGGAGGCA | 35 |
| | | CTGACATTA | |
| 24 | int5 − 44f | gccgcgaattcactagtgCTGCCAGCTC | 36 |
| | | CATGTGAC | |
| 24A | int5 − 367f | gccgcgaattcactagtgGATTTGCCCG | 37 |
| | | GTTGGAGTC | |
| 25 | int6 + 31r | ggccgcgggaattcgattAGTCACTCGC | 38 |
| | | CACTGCC | |
| 26 | ABO432f | gccgcgaattcactagtgcCACCGTGTC | 39 |
| | | CACTACTATG | |
| 27 | ABO766r | ggccgcgggaattcgattTGTAGGCCTG | 40 |
| | | GGACTGG | |
| 28 | ABO723f | gccgcgaattcactagtgGGAGGCCTTC | 41 |
| | | ACCTACG | |
| 29 | ABO1147r | ggccgcgggaattcgattCAGAGTTTAC | 42 |
| | | CCGTTCTGC | |

and amplifying the ABO gene nucleic acids.

20. (canceled)

21. (canceled)

22. A method of ABO and RHD genotyping analysis, by multiplex PCR, the method comprising contacting ABO gene and RHD gene nucleic acids from a subject with one or more of the following primer pairs 1,2; 3,4 or 4A; 5,6; 7,8 or 8A; 9 or 9A or 10 or 10A or 10B,11 or 11A; 12,13; 14 or 14A,15 15A; 18,19; 20,21; 22,23; 24 or 24A,25; 26,27; 28,29; and 30,31 from the following table, wherein the primer pairs may comprise the entire sequence shown in the table or the sequence shown in uppercase:

| | | | |
|---|---|---|---|
| | | | SEQ |
| Primer | Primer | | ID |
| no. | name | Sequence (5′-3′) | NO: |
| | | | |
|---|---|---|---|
| 1 | 101F | gccgcgaattcactagtgCCATAGAGAG | 1 |
| | | GCCAGCACAA | |
| 2 | 198R | ggccgcgggaattcgattTGCCCCTGGA | 2 |
| | | GAACCAC | |
| 3 | int1F | gccgcgaattcactagtgTGACGAGTGA | 3 |
| | | AACTCTATCTCGAT | |
| 4 | 297R | gqccgcgggaattcgattCCACCATCCC | 4 |
| | | AATACCTGAAC | |
| 4A | 296R | ggccgcgggaattcgattAGAAGTGATC | 5 |
| | | CAGCCACCAT | |
| 5 | 303F | gccgcgaattcactagtgTCCTGGCTCT | 6 |
| | | CCCTCTCT | |
| 6 | 397R | ggccgcgggaattcgattGTTGTCTTTA | 7 |
| | | TTTTTCAAAACCCT | |
| 7 | 403F | gccgcgaattcactagtgGCTCTGAACT | 8 |
| | | TTCTCCAAGGACT | |
| ·8 | 499R | ggccgcgggaattcgattCAAACTGGGT | 9 |
| | | ATCGTTGCTG | |
| 8A | 498R | ggccgcgggaattcgattATTCTGCTCA | 10 |
| | | GCCCAAGTAG | |
| 9 | 502F | gccgcgaattcactagtgCTTTGAATTA | 11 |
| | | AGCACTTCACAGA | |
| 9A | 503F | gccgcgaattcactagtgTTGAATTAAG | 12 |
| | | CACTTCACAGAGCA | |
| 10 | 5Aluint4F | gccgcgaattcactagtgAAGGACTATC | 13 |
| | (RoHar) | AGGCCACG | |
| 10A | RoHar4 | gccgcgaattcactagtgCTGAAAGGAG | 14 |
| | | GGAAACGGAC | |
| 10B | RoHar8 | gccgcgaattcactagtgGGGCAGTGAG | 15 |
| | | CTTGATAGTAGG | |
| 11 | 599R | ggccgcgggaattcgattCACCTTGCTG | 16 |
| | | ATCTTCCC | |
| 11A | 598R | ggccgcgggaattcgattTGTGACCACC | 17 |
| | | CAGCATTCTA | |
| 12 | 601F | gccgcgaattcactagtgAGTAGTGAGC | 18 |
| | | TGGCCCATCA | |
| 13 | 697R | ggccgcgggaattcgattCTTCAGCCAA | 19 |
| | | AGCAGAGGAG | |
| 14 | 702F | gccgcgaattcactagtgCTGGGACCTT | 20 |
| | | GTTAGAAATGCTG | |
| 14A | 701F | gccgcgaattcactagtgACAAACTCCC | 21 |
| | | CGATGATGTGAGTG | |
| 15 | 799R | ggccgcgggaattcgattCAAGGTAGGG | 22 |
| | | GCTGGACAG | |
| 15A | 798R | ggccgcgggaattcgattGAGGCTGAGA | 23 |
| | | AAGGTTAAGCCA | |
| 16 | 801F | gccgcgaattcactagtgCTGGAGGCTC | 24 |
| | | TGAGAGGTTGAG | |
| 17 | 899R | ggccgcgggaattcgattGGCAATGGTG | 25 |
| | | GAAGAAAGG | |
| 18 | 901F | gccgcgaattcactagtgACTGTCGTTT | 26 |
| | | TGACACACAAT | |
| 19 | 998R | ggccgcgggaattcgattTGTCACCCGC | 27 |
| | | ATGTCAG | |
| 30 | 1001F | gccgcgaattcactagtgCAAGAGATCA | 28 |
| | | AGCCAAAATCAGT | |
| 31 | 1097R | ggccgcgggaattcgattGTGGTACATG | 29 |
| | | GCTGTATTTTATTG | |
| 20 | int1 − 49f | gccgcgaattcactagtgGTGAGAGAAG | 32 |
| | | GAGGGTGAG | |
| 21 | int2 + 62r | ggccgcgggaattcgattATTGGCTGCT | 33 |
| | | GTGGTCA | |
| 22 | int3 − 33f | gccgcgaattcactagtgcCTGCTCCTA | 34 |
| | | GACTAAACTTC | |
| 23 | int4 + 52r | ggccgcgggaattcgattAAGGGAGGCA | 35 |
| | | CTGACATTA | |
| 24 | int5 − 44f | gccgcgaattcactagtgCTGCCAGCTC | 36 |
| | | CATGTGAC | |
| 24A | int5 − 367f | gccgcgaattcactagtgGATTTGCCCG | 37 |
| | | GTTGGAGTC | |
| 25 | int6 + 31r | ggccgcgggaattcgattAGTCACTCGC | 38 |
| | | CACTGCC | |
| 26 | ABO432f | gccgcgaattcactagtgcCACCGTGTC | 39 |
| | | CACTACTATG | |
| 27 | ABO766r | ggccgcgggaattcgattTGTAGGCCTG | 40 |
| | | GGACTGG | |
| 28 | ABO723f | gccgcgaattcactagtgGGAGGCCTTC | 41 |
| | | ACCTACG | |
| 29 | ABO1147r | ggccgcgggaattcgattCAGAGTTTAC | 42 |
| | | CCGTTCTGC | |

and amplifying the RHD and ABO gene nucleic acids.

23. A method according to claim 22, wherein the ABO gene and RHD gene nucleic acids are contacted with one or more of the following primer pairs 1,2; 3,4; 5,6; 7,8; 9 or 10,11; 12,13; 14,15; 18,19; 20,21; 22,23; 24,25; 26,27; 28,29; and 30,31.

24. A method according to claim 23, wherein the ABO gene and RHD gene nucleic acids are contacted with the following primer pairs 1,2; 3,4; 5,6; 7,8; 9 or 10,11; 12,13; 14,15; 18,19; 20,21; 22,23; 24,25; 26,27; 28,29; and 30,31.

25. A method according to claim 22, wherein the ABO gene and RHD gene nucleic acids are contacted with the following primer pairs 1,2; 3,4A; 5,6; 7,8A;9A or 10 or 10A or 10B,11A; 12,13; 14A,15A; 16,17;18,19; 20,21; 22,23; 24A,25; 26,27; 28,29; and 30,31.

26. A method according to claim 25, wherein the ABO gene and RHD gene nucleic acids are contacted with one or more of the following primer pairs 1,2; 3,4A; 5,6; 7,8A;9A or 10 or 10A or 10B,11A; 12,13; 14A,15A; 16,17;18,19; 20,21; 22,23; 24A,25; 26,27; 28,29; and 30,31.

27. A method of ABO and RHD genotyping analysis, by multiplex PCR, the method comprising contacting ABO gene and RHD gene nucleic acids from a subject with one or more primer from the following table wherein the primer may comprise the entire sequence shown in the table or the sequence shown in uppercase:

| | | | |
|---|---|---|---|
| | | | SEQ |
| Primer | Primer | | ID |
| no. | name | Sequence (5′-3′) | NO: |
| | | | |
|---|---|---|---|
| 1 | 101F | gccgcgaattcactagtgCCATAGAGAG | 1 |
| | | GCCAGCACAA | |
| 4 | 297R | ggccgcgggaattcgattCCACCATCCC | 4 |
| | | AATACCTGAAC | |
| 4A | 296R | ggccgcgggaattcgattAGAAGTGATC | 5 |
| | | CAGCCACCAT | |
| 5 | 303F | gccgcgaattcactagtgTCCTGGCTCT | 6 |
| | | CCCTCTCT | |
| 6 | 397R | ggccgcgggaattcgattGTTGTCTTTA | 7 |
| | | TTTTTCAAAACCCT | |
| 7 | 403F | gccgcgaattcactagtgGCTCTGAACT | 8 |
| | | TTCTCCAAGGACT | |
| 8A | 498R | ggccgcgggaattcgattATTCTGCTCA | 10 |
| | | GCCCAAGTAG | |
| 9 | 502F | gccgcgaattcactagtgCTTTGAATTA | 11 |
| | | AGCACTTCACAGA | |
| 9A | 503F | gccgcgaattcactagtgTTGAATTAAG | 12 |
| | | CACTTCACAGAGCA | |
| 10 | 5Aluint4F | gccgcgaattcactagtgAAGGACTATC | 13 |
| | (RoHar) | AGGCCACG | |
| 10A | RoHar4 | gccgcgaattcactagtgCTGAAAGGAG | 14 |
| | | GGAAACGGAC | |
| 10B | RoHar8 | gccgcgaattcactagtgGGGCAGTGAG | 15 |
| | | CTTGATAGTAGG | |
| 11 | 599R | ggccgcgggaattcgattCACCTTGCTG | 16 |
| | | ATCTTCCC | |
| 11A | 598R | ggccgcgggaattcgattTGTGACCACC | 17 |
| | | CAGCATTCTA | |
| 12 | 601F | gccgcgaattcactagtgAGTAGTGAGC | 18 |
| | | TGGCCCATCA | |
| 13 | 697R | ggccgcgggaattcgattCTTCAGCCAA | 19 |
| | | AGCAGAGGAG | |
| 14 | 702F | gccgcgaattcactagtgCTGGGACCTT | 20 |
| | | GTTAGAAATGCTG | |
| 14A | 701F | gccgcgaattcactagtgACAAACTCCC | 21 |
| | | CGATGATGTGAGTG | |
| 15 | 799R | ggccgcgggaattcgattCAAGGTAGGG | 22 |
| | | GCTGGACAG | |
| 15A | 798R | ggccgcgggaattcgattGAGGCTGAGA | 23 |
| | | AAGGTTAAGCCA | |
| 17 | 899R | ggccgcgggaattcgattGGCAATGGTG | 25 |
| | | GAAGAAAGG | |
| 18 | 901F | gccgcgaattcactagtgACTGTCGTTT | 26 |
| | | TGACACACAAT | |
| 19 | 998R | ggccgcgggaattcgattTGTCACCCGC | 27 |
| | | ATGTCAG | |
| 31 | 1097R | ggccgcgggaattcgattGTGGTACATG | 29 |
| | | GCTGTATTTTATTG | |
| 20 | int1 − 49f | gccgcgaattcactagtgGTGAGAGAAG | 32 |
| | | GAGGGTGAG | |
| 21 | int2 + 62r | ggccgcgggaattcgattATTGGCTGCT | 33 |
| | | GTGGTCA | |
| 22 | int3 − 33f | gccgcgaattcactagtgcCTGCTCCTA | 34 |
| | | GACTAAACTTC | |
| 23 | int4 + 52r | ggccgcgggaattcgattAAGGGAGGCA | 35 |
| | | CTGACATTA | |
| 24 | int5 − 44f | gccgcgaattcactagtgCTGCCAGCTC | 36 |
| | | CATGTGAC | |
| 24A | int5 − 367f | gccgcgaattcagtagtgGATTTGCCCG | 37 |
| | | GTTGGAGTC | |
| 25 | int6 + 31r | ggccgcgggaattcgattAGTCACTCGC | 38 |
| | | CACTGCC | |
| 26 | ABO432f | gccgcgaattcactagtgcCACCGTGTC | 39 |
| | | CACTACTATG | |
| 27 | ABO766r | ggccgcgggaattcgattTGTAGGCCTG | 40 |
| | | GGACTGG | |
| 28 | ABO723f | gccgcgaattcactagtgGGAGGCCTTC | 41 |
| | | ACCTACG | |
| 29 | ABO1147r | ggccgcgggaattcgattCAGAGTTTAC | 42 |
| | | CCGTTCTGC | |

and amplifying the RHD and ABO gene nucleic acids.

28.-39. (canceled)

40. A PCR primer shown in the following table, wherein the primer may comprise the entire sequence shown in the table or the sequence shown in uppercase, or a functional variant thereof:

| | | | |
|---|---|---|---|
| | | | SEQ |
| Primer | Primer | | ID |
| no. | name | Sequence (5′-3′) | NO: |
| | | | |
|---|---|---|---|
| 1 | 101F | gccgcgaattcactagtgCCATAG | 1 |
| | | AGAGGCCAGCACAA | |
| 4 | 297R | ggccgcgggaattcgattCCACCA | 4 |
| | | TCCCAATACCTGAAC | |
| 4A | 296R | ggccgcgggaattcgattAGAAGT | 5 |
| | | GATCCAGCCACCAT | |
| 5 | 303F | gccgcgaattcactagtgTCCTGG | 6 |
| | | CTCTCCCTCTCT | |
| 6 | 397R | ggccgcgggaattcgattGTTGTC | 7 |
| | | TTTATTTTTCAAAACCCT | |
| 7 | 403F | gccgcgaattcactagtgGCTCTG | 8 |
| | | AACTTTCTCCAAGGACT | |
| 8A | 498R | ggccgcgggaattcgattATTCTG | 10 |
| | | CTCAGCCCAAGTAG | |
| 9 | 502F | gccgcgaattcactagtgCTTTGA | 11 |
| | | ATTAAGCACTTCACAGA | |
| 9A | 503F | gccgcgaattcactagtgTTGAAT | 12 |
| | | TAAGCACTTCACAGAGCA | |
| 10 | 5Aluint4F | gccgcgaattcactagtgAAGGAC | 13 |
| | (RoHar) | TATCAGGCCACG | |
| 10A | RoHar4 | gccgcgaattcactagtgCTGAAA | 14 |
| | | GGAGGGAAACGGAC | |
| 10B | RoHar8 | gccgcgaattcactagtgGGGCAG | 15 |
| | | TGAGCTTGATAGTAGG | |
| 11 | 599R | ggccgcgggaattcgattCACCTT | 16 |
| | | GCTGATCTTCCC | |
| 11A | 598R | ggccgcgggaattcgattTGTGAC | 17 |
| | | CACCCAGCATTCTA | |
| 12 | 601F | gccgcgaattcactagtgAGTAGT | 18 |
| | | GAGCTGGCCCATCA | |
| 13 | 697R | ggccgcgggaattcgattCTTCAG | 19 |
| | | CCAAAGCAGAGGAG | |
| 14 | 702F | gccgcgaattcactagtgCTGGGA | 20 |
| | | CCTTGTTAGAAATGCTG | |
| 14A | 701F | gccgcgaattcactagtgACAAAC | 21 |
| | | TCCCCGATGATGTGAGTG | |
| 15 | 799R | ggccgcgggaattcgattCAAGGT | 22 |
| | | AGGGGCTGGACAG | |
| 15A | 798R | ggccgcgggaattcgattGAGGCT | 23 |
| | | GAGAAAGGTTAAGCCA | |
| 17 | 899R | ggccgcgggaattcgattGGCAAT | 25 |
| | | GGTGGAAGAAAGG | |
| 18 | 901F | gccgcgaattcactagtgACTGTC | 26 |
| | | GTTTTGACACACAAT | |
| 19 | 998R | ggccgcgggaattcgattTGTCAC | 27 |
| | | CCGCATGTCAG | |
| 31 | 1097R | ggccgcgggaattcgattGTGGTA | 29 |
| | | CATGGCTGTATTTTATTG | |
| 20 | int1 − 49f | gccgcgaattcactagtgGTGAGA | 32 |
| | | GAAGGAGGGTGAG | |
| 21 | int2 + 62r | ggccgcgggaattcgattATTGGC | 33 |
| | | TGCTGTGGTCA | |
| 22 | int3 − 33f | gccgcgaattcactagtgcCTGCT | 34 |
| | | CCTAGACTAAACTTC | |
| 23 | int4 + 52r | ggccgcgggaattcgattAAGGGA | 35 |
| | | GGCACTGACATTA | |
| 24 | int5 − 44f | gccgcgaattcactagtgCTGCCA | 36 |
| | | GCTCCATGTGAC | |
| 24A | int5 − 367f | gccgcgaattcactagtgGATTTG | 37 |
| | | CCCGGTTGGAGTC | |
| 25 | int6 + 31r | ggccgcgggaattcgattAGTCAC | 38 |
| | | TCGCCACTGCC | |
| 26 | ABO432f | gccgcgaattcactagtgcCACCG | 39 |
| | | TGTCCACTACTATG | |
| 27 | ABO766r | ggccgcgggaattcgattTGTAGG | 40 |
| | | CCTGGGACTGG | |
| 28 | ABO723f | gccgcgaattcactagtgGGAGGC | 41 |
| | | CTTCACCTACG | |
| 29 | ABO1147r | ggccgcgggaattcgattCAGAGT | 42 |
| | | TTACCCGTTCTGC | |

41. (canceled)

42. (canceled)

43. (canceled)

44. A gene chip having a plurality of attached probe sequences enabling the identification of one or more of the PCR products produced by amplification of any of the following primer pairs shown in the following table, wherein the primer pairs may comprise the entire sequence shown in the table or the sequence shown in upper case:

| | | | |
|---|---|---|---|
| | | | SEQ |
| Primer | Primer | | ID |
| no. | name | Sequence (5′-3′) | NO: |
| | | | |
|---|---|---|---|
| 1 | 101F | gccgcgaattcactagtgCCATAG | 1 |
| | | AGAGGCCAGCACAA | |
| 4 | 297R | ggccgcgggaattcgattCCACCA | 4 |
| | | TCCCAATACCTGAAC | |
| 4A | 296R | ggccgcgggaattcgattAGAAGT | 5 |
| | | GATCCAGCCACCAT | |
| 5 | 303F | gccgcgaattcactagtgTCCTGG | 6 |
| | | CTCTCCCTCTCT | |
| 6 | 397R | ggccgcgggaattcgattGTTGTC | 7 |
| | | TTTATTTTTCAAAACCCT | |
| 7 | 403F | gccqcgaattcactagtgGCTCTG | 8 |
| | | AACTTTCTCCAAGGACT | |
| 8A | 498R | ggccgcgggaattcgattATTCTG | 10 |
| | | CTCAGCCCAAGTAG | |
| 9 | 502F | gccgcgaattcactagtgCTTTGA | 11 |
| | | ATTAAGCACTTCACAGA | |
| 9A | 503F | gccgcgaattcactagtgTTGAAT | 12 |
| | | TAAGCACTTCACAGAGCA | |
| 10 | 5Aluint4F | gccgcgaattcactagtgAAGGAC | 13 |
| | (RoHar) | TATCAGGCCACG | |
| 10A | RoHar4 | gccgcgaattcactagtgCTGAAA | 14 |
| | | GGAGGGAAACGGAC | |
| 10B | RoHar8 | gccgcgaattcactagtgGGGCAG | 15 |
| | | TGAGCTTGATAGTAGG | |
| 11 | 599R | ggccgcgggaattcgattCACCTT | 16 |
| | | GCTGATCTTCCC | |
| 11A | 598R | ggccgcgggaattcgattTGTGAC | 17 |
| | | CACCCAGCATTCTA | |
| 12 | 601F | gccgcgaattcactagtgAGTAGT | 18 |
| | | GAGCTGGCCCATCA | |
| 13 | 697R | ggccgcgqgaattcgattCTTCAG | 19 |
| | | CGAAAGCAGAGGAG | |
| 14 | 702F | gccgcgaattcactagtgCTGGGA | 20 |
| | | CCTTGTTAGAAATGCTG | |
| 14A | 701F | gccgcgaattcactagtgACAAAC | 21 |
| | | TCCCCGATGATGTGAGTG | |
| 15 | 799R | ggccgcgggaattcgattCAAGGT | 22 |
| | | AGGGGCTGGACAG | |
| 15A | 798R | ggccgcgggaattcgattGAGGCT | 23 |
| | | GAGAAAGGTTAAGCCA | |
| 17 | 899R | ggccgcgggaattcgattGGCAAT | 25 |
| | | GGTGGAAGAAAGG | |
| 18 | 901F | gccgcgaattcactagtgACTGTC | 26 |
| | | GTTTTGACACACAAT | |
| 19 | 998R | ggccgcgggaattcgattTGTCAC | 27 |
| | | CCGCATGTCAG | |
| 31 | 1097R | ggccgcgggaattcgattGTGGTA | 29 |
| | | CATGGCTGTATTTTATTG | |
| 20 | int1 − 49f | gccgcgaattcactagtgGTGAGA | 32 |
| | | GAAGGAGGGTGAG | |
| 21 | int2 + 62r | ggccgcgggaattcgattATTGGC | 33 |
| | | TGCTGTGGTCA | |
| 22 | int3 − 33f | gccgcgaattcactagtgcCTGCT | 34 |
| | | CCTAGACTAAACTTC | |
| 23 | int4 + 52r | ggccgcgggaattcgattAAGGGA | 35 |
| | | GGCACTGACATTA | |
| 24 | int5 − 44f | gccgcgaattcactagtgCTGCCA | 36 |
| | | GCTCCATGTGAC | |
| 24A | int5 − 367f | gccgcgaattcactagtgGATTTG | 37 |
| | | CCCGGTTGGAGTC | |
| 25 | int6 + 31r | ggccgcgggaattcgattAGTCAC | 38 |
| | | TCGCCACTGCC | |
| 26 | ABO432f | gccgcgaattcactagtgcCACCG | 39 |
| | | TGTCCACTACTATG | |
| 27 | ABO766r | ggccgcgggaattcgattTGTAGG | 40 |
| | | CCTGGGACTGG | |
| 28 | AB0723f | gccgcgaattcactagtgGGAGGC | 41 |
| | | CTTCACCTACG | |
| 29 | ABO1147r | ggccgcgggaattcgattCAGAGT | 42 |
| | | TTACCCGTTCTGC | |
| 30 | 1001F | gccgcgaattcactagtgCAAGAG | 28 |
| | | ATCAAGCCAAAATCAGT | |
| 31 | 1097R | ggccgcgggaattcgattGTGGTA | 29 |
| | | CATGGCTGTATTTTATTG | |
| 32 | MAPH-rev | gccgcgaattcactagtg | 30 |
| 33 | MAPH-forw | ggccgcgggaattcgatt | 31 |


SEQUENCE LISTING (113 sequences)

SEQ ID NO: 1 - DNA, 38 bp, Artificial primer sequence
  Primer
gccgcgaattcactagtgccatagagaggccagcacaa

SEQ ID NO: 2 - DNA, 35 bp, Artificial primer sequence
  Primer
ggccgcgggaattcgatttgcccctggagaaccac

SEQ ID NO: 3 - DNA, 42 bp, Artificial primer sequence
  Primer
gccgcgaattcactagtgtgacgagtgaaactctatctcgat

SEQ ID NO: 4 - DNA, 39 bp, Artificial primer sequence
  primer
ggccgcgggaattcgattccaccatcccaatacctgaac

SEQ ID NO: 5 - DNA, 38 bp, Artificial primer sequence
  primer
ggccgcgggaattcgattagaagtgatccagccaccat

SEQ ID NO: 6 - DNA, 36 bp, Artificial primer sequence
  primer
gccgcgaattcactagtgtcctggctctccctctct

SEQ ID NO: 7 - DNA, 42 bp, Artificial primer sequence
  primer
ggccgcgggaattcgattgttgtctttatttttcaaaaccct

SEQ ID NO: 8 - DNA, 41 bp, Artificial primer sequence
  primer
gccgcgaattcactagtggctctgaactttctccaaggact

SEQ ID NO: 9 - DNA, 38 bp, Artificial primer sequence
  primer
ggccgcgggaattcgattcaaactgggtatcgttgctg

SEQ ID NO: 10 - DNA, 38 bp, Artificial primer sequence
  primer
ggccgcgggaattcgattattctgctcagcccaagtag

SEQ ID NO: 11 - DNA, 41 bp, Artificial primer sequence
  primer
gccgcgaattcactagtgctttgaattaagcacttcacaga

SEQ ID NO: 12 - DNA, 42 bp, Artificial primer sequence
  primer
gccgcgaattcactagtgttgaattaagcacttcacagagca

SEQ ID NO: 13 - DNA, 36 bp, Artificial primer sequence
  primer
gccgcgaattcactagtgaaggactatcaggccacg

SEQ ID NO: 14 - DNA, 38 bp, Artificial primer sequence
  primer
gccgcgaattcactagtgctgaaaggagggaaacggac

SEQ ID NO: 15 - DNA, 40 bp, Artificial primer sequence
  primer
gccgcgaattcactagtggggcagtgagcttgatagtagg

SEQ ID NO: 16 - DNA, 36 bp, Artificial primer sequence
  primer
ggccgcgggaattcgattcaccttgctgatcttccc

SEQ ID NO: 17 - DNA, 38 bp, Artificial primer sequence
  primer
ggccgcgggaattcgatttgtgaccacccagcattcta

SEQ ID NO: 18 - DNA, 38 bp, Artificial primer sequence
  primer
gccgcgaattcactagtgagtagtgagctggcccatca

SEQ ID NO: 19 - DNA, 38 bp, Artificial primer sequence
  primer
ggccgcgggaattcgattcttcagccaaagcagaggag

SEQ ID NO: 20 - DNA, 41 bp, Artificial primer sequence
  primer
gccgcgaattcactagtgctgggaccttgttagaaatgctg

SEQ ID NO: 21 - DNA, 42 bp, Artificial primer sequence
  primer
gccgcgaattcactagtgacaaactccccgatgatgtgagtg

SEQ ID NO: 22 - DNA, 37 bp, Artificial primer sequence
  primer
ggccgcgggaattcgattcaaggtaggggctggacag

SEQ ID NO: 23 - DNA, 40 bp, Artificial primer sequence
  primer
ggccgcgggaattcgattgaggctgagaaaggttaagcca

SEQ ID NO: 24 - DNA, 40 bp, Artificial primer sequence
  primer
gccgcgaattcactagtgctggaggctctgagaggttgag

SEQ ID NO: 25 - DNA, 37 bp, Artificial primer sequence
  primer
ggccgcgggaattcgattggcaatggtggaagaaagg

SEQ ID NO: 26 - DNA, 39 bp, Artificial primer sequence
  primer
gccgcgaattcactagtgactgtcgttttgacacacaat

SEQ ID NO: 27 - DNA, 35 bp, Artificial primer sequence
  primer
ggccgcgggaattcgatttgtcacccgcatgtcag

SEQ ID NO: 28 - DNA, 41 bp, Artificial primer sequence
  primer
gccgcgaattcactagtgcaagagatcaagccaaaatcagt

SEQ ID NO: 29 - DNA, 42 bp, Artificial primer sequence
  primer
ggccgcgggaattcgattgtggtacatggctgtattttattg

SEQ ID NO: 30 - DNA, 18 bp, Artificial primer sequence
  primer
gccgcgaattcactagtg

SEQ ID NO: 31 - DNA, 18 bp, Artificial primer sequence
  primer
ggccgcgggaattcgatt

SEQ ID NO: 32 - DNA, 37 bp, Artificial primer sequence
  primer
gccgcgaattcactagtggtgagagaaggagggtgag

SEQ ID NO: 33 - DNA, 35 bp, Artificial primer sequence
  primer
ggccgcgggaattcgattattggctgctgtggtca

SEQ ID NO: 34 - DNA, 39 bp, Artificial primer sequence
  primer
gccgcgaattcactagtgcctgctcctagactaaacttc

SEQ ID NO: 35 - DNA, 37 bp, Artificial primer sequence
  primer
ggccgcgggaattcgattaagggaggcactgacatta

SEQ ID NO: 36 - DNA, 36 bp, Artificial primer sequence
  primer
gccgcgaattcactagtgctgccagctccatgtgac

SEQ ID NO: 37 - DNA, 37 bp, Artificial primer sequence
  primer
gccgcgaattcactagtggatttgcccggttggagtc

SEQ ID NO: 38 - DNA, 35 bp, Artificial primer sequence
  primer
ggccgcgggaattcgattagtcactcgccactgcc

SEQ ID NO: 39 - DNA, 38 bp, Artificial primer sequence
  primer
gccgcgaattcactagtgccaccgtgtccactactatg

SEQ ID NO: 40 - DNA, 35 bp, Artificial primer sequence
  primer
ggccgcgggaattcgatttgtaggcctgggactgg

SEQ ID NO: 41 - DNA, 35 bp, Artificial primer sequence
  primer
gccgcgaattcactagtgggaggccttcacctacg

SEQ ID NO: 42 - DNA, 37 bp, Artificial primer sequence
  primer
ggccgcgggaattcgattcagagtttacccgttctgc

SEQ ID NO: 43 - DNA, 20 bp, Artificial primer sequence
  primer
ccatagagaggccagcacaa

SEQ ID NO: 44 - DNA, 17 bp, Artificial primer sequence
  primer
tgcccctggagaaccac

SEQ ID NO: 45 - DNA, 24 bp, Artificial primer sequence
  primer
tgacgagtgaaactctatctcgat

SEQ ID NO: 46 - DNA, 21 bp, Artificial primer sequence
  primer
ccaccatcccaatacctgaac

SEQ ID NO: 47 - DNA, 20 bp, Artificial primer sequence
  primer
agaagtgatccagccaccat

SEQ ID NO: 48 - DNA, 18 bp, Artificial primer sequence
  primer
tcctggctctccctctct

SEQ ID NO: 49 - DNA, 24 bp, Artificial primer sequence
  primer
gttgtctttatttttcaaaaccct

SEQ ID NO: 50 - DNA, 23 bp, Artificial primer sequence
  primer
gctctgaactttctccaaggact

SEQ ID NO: 51 - DNA, 20 bp, Artificial primer sequence
  primer
caaactgggtatcgttgctg

SEQ ID NO: 52 - DNA, 20 bp, Artificial primer sequence
  primer
attctgctcagcccaagtag

SEQ ID NO: 53 - DNA, 23 bp, Artificial primer sequence
  primer
ctttgaattaagcacttcacaga

SEQ ID NO: 54 - DNA, 24 bp, Artificial primer sequence
  primer
ttgaattaagcacttcacagagca

SEQ ID NO: 55 - DNA, 18 bp, Artificial primer sequence
  primer
aaggactatcaggccacg

SEQ ID NO: 56 - DNA, 20 bp, Artificial primer sequence
  primer
ctgaaaggagggaaacggac

SEQ ID NO: 57 - DNA, 22 bp, Artificial primer sequence
  primer
gggcagtgagcttgatagtagg

SEQ ID NO: 58 - DNA, 18 bp, Artificial primer sequence
  primer
caccttgctgatcttccc

SEQ ID NO: 59 - DNA, 20 bp, Artificial primer sequence
  primer
tgtgaccacccagcattcta

SEQ ID NO: 60 - DNA, 20 bp, Artificial primer sequence
  primer
agtagtgagctggcccatca

SEQ ID NO: 61 - DNA, 20 bp, Artificial primer sequence
  primer
cttcagccaaagcagaggag

SEQ ID NO: 62 - DNA, 23 bp, Artificial primer sequence
  primer
ctgggaccttgttagaaatgctg

SEQ ID NO: 63 - DNA, 24 bp, Artificial primer sequence
  primer
acaaactccccgatgatgtgagtg

SEQ ID NO: 64 - DNA, 19 bp, Artificial primer sequence
  primer
caaggtaggggctggacag

SEQ ID NO: 65 - DNA, 22 bp, Artificial primer sequence
  primer
gaggctgagaaaggttaagcca

SEQ ID NO: 66 - DNA, 22 bp, Artificial primer sequence
  primer
ctggaggctctgagaggttgag

SEQ ID NO: 67 - DNA, 19 bp, Artificial primer sequence
  primer
ggcaatggtggaagaaagg

SEQ ID NO: 68 - DNA, 21 bp, Artificial primer sequence
  primer
actgtcgttttgacacacaat

SEQ ID NO: 69 - DNA, 17 bp, Artificial primer sequence
  primer
tgtcacccgcatgtcag

SEQ ID NO: 70 - DNA, 23 bp, Artificial primer sequence
  primer
caagagatcaagccaaaatcagt

SEQ ID NO: 71 - DNA, 24 bp, Artificial primer sequence
  primer
gtggtacatggctgtattttattg

SEQ ID NO: 72 - DNA, 19 bp, Artificial primer sequence
  primer
gtgagagaaggagggtgag

SEQ ID NO: 73 - DNA, 17 bp, Artificial primer sequence
  primer
attggctgctgtggtca

SEQ ID NO: 74 - DNA, 20 bp, Artificial primer sequence
  primer
ctgctcctagactaaacttc

SEQ ID NO: 75 - DNA, 19 bp, Artificial primer sequence
  primer
aagggaggcactgacatta

SEQ ID NO: 76 - DNA, 18 bp, Artificial primer sequence
  primer
ctgccagctccatgtgac

SEQ ID NO: 77 - DNA, 19 bp, Artificial primer sequence
  primer
gatttgcccggttggagtc

SEQ ID NO: 78 - DNA, 17 bp, Artificial primer sequence
  primer
agtcactcgccactgcc

SEQ ID NO: 79 - DNA, 19 bp, Artificial primer sequence
  primer
caccgtgtccactactatg

SEQ ID NO: 80 - DNA, 17 bp, Artificial primer sequence
  primer
tgtaggcctgggactgg

SEQ ID NO: 81 - DNA, 17 bp, Artificial primer sequence
  primer
ggaggccttcacctacg

SEQ ID NO: 82 - DNA, 19 bp, Artificial primer sequence
  primer
cagagtttacccgttctgc

SEQ ID NO: 83 - DNA, 454 bp, Homo sapiens
cttccgtgttaactccatagacaggccagcacagccagccttgcagcctgagataaggcctttggcgggtgtctcccctatcgctccctcaagccctcaagtaggtgttggagagaggggtgatgcctggtgctggtggaacccctgcacagagacggacacaggatgagctctaagtacccgcggtctgtccggcgctgcctgcccctctgcgccctaacactggaagcagctctcattctcctcttctatttttttacccactatgacgcttccttagaggatcaaaaggggctcgtggcatcctatcaaggtgagagttcattggaacagtggtcacaggagcaaatagcaggggcaggggcgggggaggcctatggttctccaggggcacagatgttcctttctacaaaatcccgaggaaaagattcccccatcttcttccgtagattgc

SEQ ID NO: 84 - DNA, 455 bp, Homo sapiens
cttccgtgttaactccatagagaggccagcacaaccagccttgcagcctgagataaggcctttggcgggtgtctcccctatcgctccctcaagccctcaagtaggtgttggagagaggggtgatgcctggtgctggtggaacccctgcacagagacggacacaggatgagctctaagtacccgcggtctgtccggcgctgcctgcccctctgggccctaacactggaagcagctctcattctcctcttctatttttttacccactatgacgcttccttagaggatcaaaaggggctcgtggcatcctatcaaggtgagagttcattggaaaagtggtcacaggagcaaatagcaggggcaggggcgggggaggcctgtggttctccaggggcacagatgttcctttctacaaaatcccaaggaaaaagattcccccatcttcttccgtagattgc

SEQ ID NO: 85 - DNA, 1365 bp, Homo sapiens
ttgaacccaggaggcagaggttgcagtgagccaagatctcgccactgtactccagcctgggtgacaagagtgaaactctatctcaaaattaaaaaaaaaaaatcttagctctacccaccggggcaagttacataacgcctctgtgccttggttttcatatctgtaaaatggtgacagtaacagcacccatgtcaaagtgtggttgtgagaacgaaacaagatagtctatgtaaagtgattaaaacagcgtaggcacatggtaaacgcttaggaaatgtaggctgttataaagctcagagatgttaagtaactagatcaagaccacacagttagagggtgccacagtcttgatttgaacccaaatttgtctcgttctggagctcaagctgctaaccctttttcaaaactggaattaaaccaaagtgctcaccctccgctttgctgggcccctccctgccctcaggtgcatctcttccactcacctgccacagcagcctctgctcagggtctgagactgggaaaggtgagggctacccaggtggccctgatgttttctgccagccagctcaccaggtccctcgcagcaggcggcaaagggagggaggtttgctgtgaagattatgtggttcccaacaacaagagcactgggcctatctctgccctctcttttctgtgtgtcctgggacaagtcacttggcttctgtggctttattttctcatgtgcccagccagggggttggccctcatatgcaataacagcagcaatgacctttactgagtgtccatgtgcatcaagcacgtgtactttacacttgttcttattattaggtttaataatagaataattgccacatttactgagcactcattatgggccaggccctgccctaagtgcttaattagctttagctcctctaatccttaccttatccccacacggcatgttatgttatccccattattcagttgagaacattgaggctcaaagaggcaaagtaacttgaccaaatacttgtaaacgatcttgcatgccccttccagctgccatttagtaagactctaatttcataccaccctaaatctcgtctgcttccccctcctccttctcaccatctccccaccgagcagtcggccaagatctgaccgtgatggcggcccttggcttgggcttcctcacctcaaatttccggagacacagctggagcagtgtggccttcaacctcttcatgctggcgcttggtgtgcagtgggcaatcctgctggacggcttcctgagccagttccctcctgggaaggtggtcatcacactgttcaggtattgggatggtggctggatcacttctgggtcatagagggaatggaccc

SEQ ID NO: 86 - DNA, 1362 bp, Homo sapiens
ttgaacccaggaggcagaggttgcagtgagccaagatcttgccactgtactccagcctgggtgacgagtgaaactctatctcgatattaaaaaaaaaaatcttagctctacccaccggggcaagttacgtaacgcctctgtgccttggttttcatatctgtaaaatggtgacagtaacagcacccacgtcaaagtgtggttgtgagaacgaaacaagatagtctatgtaaagtgattaaaacagcgtaggcacatggtaaacgcttaggaaatgtaggctgttataaagctcagagatgttaagtaactagatcaagatcacacagttagagggtgccagagtcctgatttgaacccaagtttgtctcgttctggagctcaagctgctaaccctttttcaaaactggaattaaaccaaagtgctcaccctccgctttgctgggcccctccctgccctcaggtgcgtctcttccactcacctgccacagcagcctctgctcagggtctgagaccgggaaaggtgagggctacccaggtggccctgatgttttctgccagccagctcaccaggtccctcgcagcaggcggcaaagggagggaggtttgctgtgaagattatgtggttcccaacaacaagagcgctgggcctatctctgccctctcttttctgtgtgtcctgggacaagtcacttggcttctgtggcttcattttctcatgtgcccagccagggggttggccctcatatgcaataacagcagcaatgacctttactgagtgtccatgtgcgtcaagcacgtgtgctttacacttgttcttattattaggtttaataatagaataattgccacatttactgagcactcattatgggccaggccctgccctaagtgcttaattagctttagctcctctaatccttatcttatccccacacggcatgttatgttatccccattattcagttgagaacattgaggctcaaagaggcaaagtaacttgaccaaatacttgtaaacgatcttgcatgccccttccagctgccatttagtaagactctaatttcataccaccctaaatctcgtctgcttccccctcgtccttctcgccatctccccaccgagcagttggccaagatctgaccgtgatggcggccattggcttgggcttcctcacctcgagtttccggagacacagctggagcagtgtggccttcaacctcttcatgctggcgcttggtgtgcagtgggcaatcctgctggacggcttcctgagccagttcccttctgggaaggtggtcatcacactgttcaggtattgggatggtggctggatcacttctgggtcatagagggaatggaccc

SEQ ID NO: 87 - DNA, 325 bp, Homo sapiens
ccttctcagtcatcctggctctccttctcacccccagtattcggctggccaccatgagtgctatgtcggtgctgatctcagcgggtgctgtcttggggaaggtcaacttggcgcagttggtggtgatggtgctggtggaggtgacagctttaggcaccctgaggatggtcatcagtaatatcttcaacgtgagtcatggtgctgggaggagggacctgggagaaaagggccaaaagctccatttggtggggcttccggggttttgaaaaataaagacaacctgtaatcccagctacttgggaggttgaggagggaagatcacttg

SEQ ID NO: 88 - DNA, 325 bp, Homo sapiens
ccttctcagtcgtcctggctctccctctctcccccagtattcggctggccaccatgagtgctttgtcggtgctgatctcagtggatgctgtcttggggaaggtcaacttggcgcagttggtggtgatggtgctggtggaggtgacagctttaggcaacctgaggatggtcatcagtaatatcttcaacgtgagtcatggtgctgggaggagggacctgggagaaaagggccaaaagctccatttggtggggtttccagggttttgaaaaataaagacaacctgtaatcccagctacttgggaggttgaggagggaagatcacttg

SEQ ID NO: 89 - DNA, 390 bp, Homo sapiens
tgggttgggctgggtaagctctgaacaccagtctcgtggcttcaagtcacacctcctaagtgaagctctgaactttctccaaggaccatcagggctttcccctgggcagaggatgccgacactcactgctcttactgggttttattgcagacagactaccacatgaacctgaggcacttctacgtgttcgcagcctattttgggctgactgtggcctggtgcctgccaaagcctctacccaagggaacggaggataatgatcagagagcaacgatacccagtttgtctgccatgctgggtaaggacaaggtggggtgagtggtctcatacttgggctgagcagaatggctcagaaaaggctctggctgaaaaaatctccctcctttacca

SEQ ID NO: 90 - DNA, 389 bp, Homo sapiens
tgggttgggctgggtaagctctgaacaccagtctcatggcttcaagtcacacctcctaagtgaagctctgaactttctccaaggactatcagggcttgccccgggcagaggatgccgacactcactgctcttactgggttttattgcagacagactaccacatgaacatgatgcacatctacgtgttcgcagcctattttgggctgtctgtggcctggtgcctgccaaagcctctacccgagggaacggaggataaagatcagacagcaacgatacccagtttgtctgccatgctgggtaaggacaaggtggggtgagtggtctcctacttgggctgagcagaatggctcagaaaaggctctggctgaaaaaatctccctcctttacca

SEQ ID NO: 91 - DNA, 1300 bp, Homo sapiens
catacctttgaattaagcacttccttttagggacctctcttcattaatatccactagaaaggagagactcattatgtgtgagtttcaataagtttatccaatccctttgttttcaactgaaaggagggaaacggacaagtgaagaaggtagggcccaggagtgaaggaacaagggtgggaatagtaataatgttgtactttgaaaatctactgggaaaatgatgaacttagactgctgggagaggctaatagaaaatcgggcagtgagcttgatagtaggcaaaggactatcaggccacggggtcaagttaaagcagcacattcattaaaaaaaaaataaataagcgtttgggccaggcgtggtggctcaagcctgtaatcccagcactttgggaggccaaggtgggtggatcacctgaggtcaggagttcgagaccagcctggccaacagggcgaaaccccatctctactaaaaatacaaacaaattagctgggcatggtggtgcacgcctgtaatcccagctacttgggaggctgaggcaggagaatcttttgaatccaggtggtggaggttgcagtgagccaagatcgcgccactgcactccagcctgggcaacagagcaagagtccatctcaattaaaaagaaaaaaaaattaaaataagcatttgaccatcacagagcaggttcaggaggcctggggtatgcagatttcaaccctcttggcctttgtttccttgtctgtaaaatgtggttagctggtatcagcttgagagctcggaggggagacgtgacttccccatctaactctaagtgacaaggctgagactctccagccctaggattctcatccaaaacccctcgaggctcagacctttggagcaggagtgtgattctggccaaccaccctctctggcccccaggcgccctcttcttgtggatgttctggccaagtgtcaactctgctctgctgagaagtccaatccaaaggaagaatgccatgttcaacacctactatgctctagcagtcagtgtggtgacagccatctcagggtcatccttggctcacccccaaaggaagatcagcatggtgagcagggcgctgcccttgggcagcacttgggtctaacaggactagcacacatatttatgcccctccccaccccagggccagcgtgggttgggagaggacatgccgggtggtggagctgtgcctgcctctacagtggagctctaggaagaatgctgggtggtcacagggggcctgggactcaggagactgtccagtgatcaaaggcttt

SEQ ID NO: 92 - DNA, 647 bp, Homo sapiens
catacctttgaattaagcacttcacagagcaggttcaggaggcctggggtatgcagatttcaaccctcttggcctttgtttccttgtctgtaaaatgtggttagctggtatcagcttgagagctcggaggggagacgtgacttccccatctaactctaagtgacaaggctgagactctccagccctaggattctcatccaaaacccctcgaggctcagacctttggagcaggagtgtgattctggccaaccaccctctctggcccccaggcgccctcttcttgtggatgttctggccaagtttcaactctgctctgctgagaagtccaatcgaaaggaagaatgccgtgttcaacacctactatgctgtagcagtcagcgtggtgacagccatctcagggtcatccttggctcacccccaagggaagatcagcaaggtgagcagggcgctgcccttgggcagcacttgggtctaacaggactagcacacatatttatgcccctccccaccccagggccagcgtgggttgggagagggcatgccgggtggtggagctgtgcctgcctctacagtggagctctaggtagaatgctgggtggtcacagtgggcctgggactcaggagactgtccagtgatcaaaggcttt

SEQ ID NO: 93 - DNA, 390 bp, Homo sapiens
cctaagaggcagtagtgagctggcccaccgtgtccactgatgaaggacacgtagccccaacacaggggagaggtggtttcaggatcagcaaagcagggaggatgttacagggttgccttgttcccagcgtgctggtcacttgcagcaagatggtgttctctctctaccttgcttcctttacccacacgctatttctttgcagacttatgtgcacagtgcggtgttggcaggaggcgtggctgtgggtacctcgtgtcacctgatcccttctccgtggcttgccatggtgctgggtcttgtggctgggctgatctccatcgggggagccaagtgcctgccggtaagaaactagacaactaatgctctctgctttggctgaaggccagcagg

SEQ ID NO: 94 - DNA, 390 bp, Homo sapiens
cctaagaggcagtagtgagctggcccatcatgtccactgatgaaggacacgtagccccaacacaggggagaagtggtttcaggatcagcaaagcagggaggatgttacagggttgccttgttcccagcgtgctggtcacttgcagcaagatggtgttctctctctaccttgcttcctttacccacacgctatttctttgcagacttatgtgcacagtgcggtgttggcaggaggcgtggctgtgggtacctcgtgtcacctgatcccttctccgtggcttgccatggtgctgggtcttgtggctgggctgatctccgtcgggggagccaagtacctgccggtaagaaactagacaactaacctcctctgctttggctgaaggccagcagg

SEQ ID NO: 95 - DNA, 650 bp, Homo sapiens
gtgcctacactagacccttgctactcatagtgtggtccgtagatgagcagcattggcatcacctgggaccttgttagaaatgctcttagaccccaccccacatccactaaagccagctcttcatttcaacaaactccccattgatgtgagtacacattcaagtctgagaagggcttctttgaggtgagccttagtgcccatccccatttggtggcgccggataccaagggtgtgtgaaaggggtgggtagggaatatgggtctcacctgccaatctgcttataataacacttgtccacaggtgtgttgtaaccgagtgctggggattcaccacatctccgtcatgcactccatcttcagcttgctgggtctgcttggagagatcacctacattgtgctgctggtgcttcatactgtctggaacggcaatggcatgtgggtcactgggcttaccccccatccccttaacactcccctccaactcaggaagaaatgtgtgcagagtccttagctggggcgtgtgcactcggggccaggtgctcagtaggcttcggtgaatatttgttggctgatttattcagaaattatgtccagcccctaccttggatggatttatcacctctccaggccacctcttctttccaaatagga

SEQ ID NO: 96 - DNA, 650 bp, Homo sapiens
gtgtctacactagacccttgctactcatagtgtggtccgtagaccagcagcattggcatcacctgggaccttgttagaaatgctgttagaccccaccccacatccactaaagccagctcttcatttcaacaaactccccgatgatgtgagtgcacattcaagtctgagaagggcttctttgaggtgagccttagtgcccatccccctttggtggccccggataccaagggtgtgtgaaaggggtgggtagggaatatgggtctcacctgccaatctgcttataataacacttgtccacaggggtgttgtaaccgagtgctggggattccccacagctccatcatgggctacaacttcagcttgctgggtctgcttggagagatcatctacattgtgctgctggtgcttgataccgtcggagccggcaatggcatgtgggtcactgggcttaccccccatccccttaacactcccctccaactcaggaagaaatgtgtgcagagtccttagctggggcgtgtgcactcggggccaggtgctcagtaggcttcggtgaatatttgttggctgatttattcagaaattctgtccagcccctaccttggatggatttatcacctctccaggccacctcttctttccaaataggg

SEQ ID NO: 97 - DNA, 780 bp, Homo sapiens
ggaccttgttagaaatgctcttagaccccaccccacatccactaaagccagctcttcatttcaacaaactccccattgatgtgagtacacattcaagtctgagaagggcttctttgaggtgagccttagtgcccatccccatttggtggcgccggataccaagggtgtgtgaaaggggtgggtagggaatatgggtctcacctgccaatctgcttataataacacttgtccacaggtgtgttgtaaccgagtgctggggattcaccacatctccgtcatgcactccatcttcagcttgctgggtctgcttggagagatcacctacattgtgctgctggtgcttcatactgtctggaacggcaatggcatgtgggtcactgggcttaccccccatccccttaacactcccctccaactcaggaagaaatgtgtgcagagtccttagctggggcgtgtgcactcggggccaggtgctcagtaggcttcggtgaatatttgttggctgatttattcagaaattatgtccagcccctaccttggatggatttatcacctctccaggccacctcttctttccaaataggaccacctaggtatagaccaaagacacgaaatcttctgtgaccccacaaacacagagcaggtcaaataggcccaagccaattgagactgtggttcaggtcgtgatgcagagctttgctgtggacgtgctcccactgcgtactagctgggcatgcggcttaacctttctcagcctcagtcgcccccttgtaaatggag

SEQ ID NO: 98 - DNA, 780 bp, Homo sapiens
ggaccttgttagaaatgctgttagaccccaccccacatccactaaagccagctcttcatttcaacaaactccccgatgatgtgagtgcacattcaagtctgagaagggcttctttgaggtgagccttagtgcccatccccctttggtggccccggataccaagggtgtgtgaaaggggtgggtagggaatatgggtctcacctgccaatctgcttataataacacttgtccacaggggtgttgtaaccgagtgctggggattccccacagctccatcatgggctacaacttcagcttgctgggtctgcttggagagatcatctacattgtgctgctggtgcttgataccgtcggagccggcaatggcatgtgggtcactgggcttaccccccatccccttaacactcccctccaactcaggaagaaatgtgtgcagagtccttagctggggcgtgtgcactcggggccaggtgctcagtaggcttcggtgaatatttgttggctgatttattcagaaattctgtccagcccctaccttggatggatttatcacctctccaggccacctcttctttccaaatagggccacctaggtatagaccaaagacacgaaatcttttgtgatcccacaaacacagagcaggtcaaataggcccaagccaattgagactgtggttcaggtcgtgatgcagagctttgctgtggacgtgctcccactgcgtactagctgggcatgtggcttaacctttctcagcctcagtcgccccattgtaaatggag

SEQ ID NO: 99 - DNA, 520 bp, Homo sapiens
cagaaaaaaaaaaaaaaaaaagagagagagagaaaactggaggctctgagaggttaaaggacttgcccagggtcttgcagctagtaagtgacagagctgggacttgagcttgggttttctgactcctggtctggttcattatccatgaggtgctgggaactaaaataagccacaatcttggaatctccgtcgcctccctccctcccacatgtctgcgtggctttttgggaaaatgccaggggaatgtaccagccagggagaggacccttgttttcctcatggcccttcctggcaatggcactactgacaccgacagtcctttttgtccctgatgacctctgctgcctgatgcccaagtgaccacctctgctttgtcatttctaggattggcttccaggtcctcctcagcattggggaactcagcttggccatcgtgatagctctcatgtctggtctcctgacaggtcagtgtgaggccacctttcttccaccattgccaggacacagcacccacgtccag

SEQ ID NO: 100 - DNA, 519 bp, Homo sapiens
cagaaaaaaaaaaaaaaaaagagagagagagaaaactggaggctctgagaggttgagggacttgcccagggtcttgcagctagtaagtgacagagctgggacttgagcttgggttttctgactcctggtctggttcattatccatgaggtgctgggaactaaaataagccacaatcttggaatctccgtcgcctccctccctcccacatgtctgcgtggctttttgggaaaatgccaggggaatgtaccagccagggagaggacccttgttttcctcatggcccttcctggcaatggcactactgacaccgacagtcctttttgtccctgatgacctctgctgcctgatgcccaagtgaccacctctgctttgtcatttctaggattggcttccaggtcctcctcagcattggggaactcagcttggccatcgtgatagctctcacgtctggtctcctgacaggtcagtgtgaggccacctttcttccaccattgccaggacacagcacccacgtccag

SEQ ID NO: 101 - DNA, 520 bp, Homo sapiens
gaaaaaggatttctgttgagacactgtcgttttgacacacacaatattttgattaatcttgagattaaaaatcctgtgctccaaatcttttaacattaaattatgcatttaaacaggtttgctcctaaatctcaaaatatggaaagcacctcatgtggctaaatattttgatgaccaagttttctggaaggtaagatttttcacctattaacgtgatagattttgagtgcatgaacttaaaaacatacctgggtatatatgttgacttgctgtttatgagtaaaacaaaaacaaaaatggagtaaggagcattgcaggaggaactagaggagaaacaaatccatgatatgcatgtgtgtgggggagggtggcggggaggtggtaaaggtcaccatttccctgatacctcaaattcattcagagtcagggatgagacagctttcactggccacacttcccctcccgctatctgcagtcctcagcgtagccaaatagtttgacatgcgggtgacagaacccc

SEQ ID NO: 102 - DNA, 518 bp, Homo sapiens
gaaaaaggatttctgttgagatactgtcgttttgacacacaatatttcgattaatcttgagattaaaaatcctgtgctccaaatcttttaacattaaattatgcatttaaacaggtttgctcctaaatcttaaaatatggaaagcacctcatgaggctaaatattttgatgaccaagttttctggaaggtaagatttttcacctattaacgtgatagattttgagtgcatgaacttaaaaacatacctgagtatatatgttgacttgctgtttatgagtaaaacaaaaacaaaaatggagtaaggagcattgcaggaggaactagaggagaaacaaatccatgatatgcatgtgtgtgggggagggtggcggggaggtggtaaaggtcaccatttccctgatacctcaaattcattcagagtcagggatgagacagctttcactggccacacttcccctccccctatctgcagtcctcagcgtagccaaatagtctgacatgcgggtgacagaacccc

SEQ ID NO: 103 - DNA, 373 bp, Homo sapiens
ctgtttcaagagatcaagccaaaatcagtatgtgggttcatctgcaataaaaatgtttgttttgcttttacagtttcctcatttggctgttggattttaagcaaaagcatccaagaaaaacaaggcctgttcaaaaacaagacaacttcctctcactgttgcctgcatttgtacgtgagaaacgctcatgacagcaaagtctccttatgtataatgaaacaaggtcagagacagatttgatattaaaaaattaaagactaaaaacttagtttaagagtcaatttaataagtttaaaataaatgtttagtttcattaggatgatgctatcaatattttcttggttacagacacattattaaagttttgggttaa

SEQ ID NO: 104 - DNA, 381 bp, Homo sapiens
ctgtttcaagagatcaagccaaaatcagtatgtgggttcatctgcaataaaaatgtttgttttgcttttacagtttcctcatttggctgttggattttaagcaaaagcatccaagaaaaacaaggcctgttcaaaaacaagacaacttcctctcactgttgcctgcatttgtacgtgagaaacgctcatgacagcaaagtctccaatgttcgcgcaggcactggagtcagagaaaatggagttgaatcctttctctgccactctttgaggagaatctcaccatttattatgcactgtagaatacaacaataaaatacagccatgtaccacataacaacatcttggtaaacaacagactgcatatatgatggtggtcatcca

SEQ ID NO: 105 - DNA, 181 bp, Homo sapiens
gtgagagaaggagggtgagtgatgtgatttttctactcctgttttccaggaaaaccaaaatgccacgcacttcgacctatgatccttttcctaataatgcttgtcttggtcttgtttgggtaagacacatttgaccatcgaggctggcctggtttggggagaagtgaccacagcagccaat

SEQ ID NO: 106 - DNA, 115 bp, Homo sapiens
cctgctcctagactaaacttcatctcctgtgttctcattctgcagcatggctgttagggaacctgaccatctgcagcgcgtctcgttgccaaggtataatgtcagtgcctccctt

SEQ ID NO: 107 - DNA, 227 bp, Homo sapiens
ctgccagctccatgtgaccgcacgcctctctccatgtgcagtaggaaggatgtcctcgtggtgaccccttggctggctcccattgtctgggagggcacattcaacatcgacatcctcaacgagcagttcaggctccagaacaccaccattgggttaactgtgtttgccatcaagaagtaagtcagtgaggtggccgagggtagagacccaggcagtggcgagtgact

SEQ ID NO: 108 - DNA, 335 bp, Homo sapiens
ccaccgtgtccactactatgtcttcaccgaccagccggccgcggtgccccgcgtgacgctggggaccggtcggcagctgtcagtgctggaggtgcgcgcctacaagcgctggcaggacgtgtccatgcgccgcatggagatgatcagtgacttctgcgagcggcgcttcctcagcgaggtggattacctggtgtgcgtggacgtggacatggagttccgcgaccacgtgggcgtggagatcctgactccgctgttcggcaccctgcaccccggcttctacggaagcagccgggaggccttcacctacgagcgccggccccagtcccaggcctaca

SEQ ID NO: 109 - DNA, 425 bp, Homo sapiens
ggaggccttcacctacgagcgccggccccagtcccaggcctacatccccaaggacgagggcgatttctactacctgggggggttcttcggggggtcggtgcaagaggtgcagcggctcaccagggcctgccaccaggccatgatggtcgaccaggccaacggcatcgaggccgtgtggcacgacgagagccacctgaacaagtacctgctgcgccacaaacccaccaaggtgctctcccccgagtacttgtgggaccagcagctgctgggctggcccgccgtcctgaggaagctgaggttcactgcggtgcccaagaaccaccaggcggtccggaacccgtgagcggctgccaggggctctgggagggctgccggcagccccgtccccctcccgcccttggttttagcagaacgggtaaactctg

SEQ ID NO: 110 - DNA, 181 bp, Homo sapiens; identical to SEQ ID NO: 105

SEQ ID NO: 111 - DNA, 115 bp, Homo sapiens; identical to SEQ ID NO: 106

SEQ ID NO: 112 - DNA, 374 bp, Homo sapiens
gatttgcccggttggagtcgcatttgcctctggttggtttcccggggaagggcggctgcctctggaagggtggtcagaggaggcagaagctgagtggagtttccaggtgggggcggccgtgtgccagaggcgcatgtgggtggcaccctgccagctccatgtgaccgcacgcctctctccatgtgcagtaggaaggatgtcctcgtggtgaccccttggctggctcccattgtctgggagggcacattcaacatcgacatcctcaacgagcagttcaggctccagaacaccaccattgggttaactgtgtttgccatcaagaagtaagtcagtgaggtggccgagggtagagacccaggcagtggcgagtgact

SEQ ID NO: 113 - DNA, 716 bp, Homo sapiens
ccaccgtgtccactactatgtcttcaccgaccagccggccgcggtgccccgcgtgacgctggggaccggtcggcagctgtcagtgctggaggtgcgcgcctacaagcgctggcaggacgtgtccatgcgccgcatggagatgatcagtgacttctgcgagcggcgcttcctcagcgaggtggattacctggtgtgcgtggacgtggacatggagttccgcgaccacgtgggcgtggagatcctgactccgctgttcggcaccctgcaccccggcttctacggaagcagccgggaggccttcacctacgagcgccggccccagtcccaggcctacatccccaaggacgagggcgatttctactacctgggggggttcttcggggggtcggtgcaagaggtgcagcggctcaccagggcctgccaccaggccatgatggtcgaccaggccaacggcatcgaggccgtgtggcacgacgagagccacctgaacaagtacctgctgcgccacaaacccaccaaggtgctctcccccgagtacttgtgggaccagcagctgctgggctggcccgccgtcctgaggaagctgaggttcactgcggtgcccaagaaccaccaggcggtccggaacccgtgagcggctgccaggggctctgggagggctgccggcagccccgtccccctcccgcccttggttttagcagaacgggtaaactctg